(19) United States
(12) Patent Application Publication
(10) Pub. No.: US 2011/0213248 A1
(43) Pub. Date: Sep. 1, 2011

(54) ULTRASONIC TREATMENT APPARATUS

(75) Inventors: Miyuki MURAKAMI, Hino-shi (JP); Hiroshi TSURUTA, Sagamihara-shi (JP); Yoshiharu ISHIBASHI, Hino-shi (JP)

(73) Assignee: OLYMPUS CORPORATION, Tokyo (JP)

(21) Appl. No.: 13/027,626

(22) Filed: Feb. 15, 2011

(30) Foreign Application Priority Data
Feb. 26, 2010 (JP) 2010-042570

(51) Int. Cl. A61B 8/14; A61N 7/00

(52) U.S. Cl. 600/439; 601/2

(57) ABSTRACT

An ultrasonic treatment apparatus includes a sound source, a frequency setter and a drive signal generator. The sound source emits ultrasonic waves. The ultrasonic waves are finite amplitude acoustic waves including a first signal frequency and a second signal frequency. The frequency setter sets the first signal frequency and the second signal frequency in accordance with a target position of a treatment object with respect to the sound source. The drive signal generator is configured to generate a drive signal and to drive the sound source. The drive signal is a signal which causes the sound source to emit the ultrasonic waves.

### CROSS-REFERENCE TO RELATED APPLICATIONS

This application is based upon and claims the benefit of priority from prior Japanese Patent Application No, 2010-042570, filed Feb. 26, 2010, the entire contents of which are incorporated herein by reference.

### BACKGROUND OF THE INVENTION

1. Field of the Invention

The present invention relates to an ultrasonic treatment apparatus.

2. Description of the Related Art

According to one general treatment technique, ultrasonic waves are applied to a target tissue by an ultrasonic radiation device to destroy cells constituting the tissue or to heat and coagulate the tissue. An ultrasonic treatment apparatus for use in such a treatment is disclosed in, for example, Jpn. Pat. Appln. KOKAI Publication No. 2004-113254. It is known that the propagation distance of ultrasonic waves varies depending on the frequency of the ultrasonic waves. For example, the above-mentioned publication also discloses that ultrasonic wave generating elements are exchanged to change the frequency of ultrasonic waves to be radiated depending on a position to be irradiated with ultrasonic waves.

It is known that in the above-mentioned ultrasonic irradiation treatment, if microbubbles used as an ultrasonic contrast agent are provided to the target tissue and the microbubbles are vibrated or burst by ultrasonic irradiation, the efficiency of this treatment increases as a result of a cavitation effect.

### BRIEF SUMMARY OF THE INVENTION

According to an aspect of the invention, an ultrasonic treatment apparatus includes a sound source which emits ultrasonic waves, the ultrasonic waves being finite amplitude acoustic waves including a first signal frequency and a second signal frequency; a frequency setter which sets the first signal frequency and the second signal frequency in accordance with a target position of a treatment object with respect to the sound source; and a drive signal generator configured to generate a drive signal and to drive the sound source, the drive signal being a signal which causes the sound source to emit the ultrasonic waves.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing a configuration example of an ultrasonic treatment apparatus according to a first embodiment of the present invention;

FIG. 2 is a plane view showing an example of a cMUT array constituting an ultrasonic emitter according to the first embodiment of the present invention;

FIG. 3A is a sectional view showing an example of a cMUT constituting the ultrasonic emitter according to the first embodiment of the present invention;

FIG. 3B is a plane view showing an example of a cMUT constituting the ultrasonic emitter according to the first embodiment of the present invention;

FIG. 4 is a schematic graph illustrating the frequency characteristics of an output of the ultrasonic treatment apparatus according to the first embodiment of the present invention;

FIGS. 5 and 6 are sectional views showing other examples of the cMUT constituting the ultrasonic emitter according to the first embodiment of the present invention;

FIG. 7 is a sectional view showing another example of the cMUT constituting the ultrasonic emitter according to the first embodiment of the present invention;

FIG. 8 is a diagram showing an example of tables which are stored in an output information storage according to the first embodiment of the present invention and which represent the relation between the depths of target positions and output frequencies;

FIG. 9 is a diagram showing a configuration example of an endoscopic ultrasonic diagnostic treatment apparatus according to a second embodiment of the present invention;

FIG. 10 is a block diagram showing a configuration example of one form of an ultrasonic wave control unit and its associated components in the endoscopic ultrasonic diagnostic treatment apparatus according to the second embodiment of the present invention;

FIG. 11 is a schematic diagram illustrating an image acquired by the ultrasonic apparatus according to the second embodiment of the present invention;

FIG. 12A is a plane view showing an example of a cMUT array constituting an ultrasonic emitter according to a first modification of the second embodiment of the present invention;

FIG. 12B is a sectional view showing an example of the cMUT array constituting an ultrasonic emitter according to the first modification of the second embodiment of the present invention;

FIG. 13 is a schematic diagram illustrating an image acquired by the ultrasonic apparatus according to the first modification of the second embodiment of the present invention;

FIG. 14 is a schematic diagram illustrating the relation between the phase and wave front of ultrasonic waves output by an ultrasonic emitter according to the second embodiment of the present invention;

FIG. 15 is a diagram showing a configuration example of a tip portion according to a second modification of the second embodiment of the present invention;

FIG. 16 is a block diagram showing a configuration example of an ultrasonic treatment apparatus according to a third embodiment of the present invention;

FIG. 17 is a schematic diagram illustrating radiation ranges of ultrasonic waves generated by an ultrasonic treatment apparatus according to a fourth embodiment of the present invention and also illustrating a difference tone generated in a region where the radiation ranges are superposed on each other;

FIG. 18 is a block diagram showing a configuration example of an ultrasonic treatment apparatus according to a fourth embodiment of the present invention;

FIGS. 19A and 19B are schematic diagrams illustrating the radiation ranges of the ultrasonic waves generated by the ultrasonic treatment apparatus according to the fourth embodiment of the present invention and also illustrating the region where the radiation ranges are superposed on each other and the difference tone is thus generated;

FIG. 20A is a plane view showing an example of a cMUT array constituting an ultrasonic emitter according to a first modification of the fourth embodiment of the present invention;

FIG. 20B is a sectional view showing an example of the cMUT array constituting an ultrasonic emitter according to the first modification of the fourth embodiment of the present invention;

FIGS. 21A and 21B are schematic diagrams illustrating radiation ranges of ultrasonic waves generated by an ultrasonic treatment apparatus according to the first modification of the fourth embodiment of the present invention and also illustrating a region where the radiation ranges are superposed on each other and a difference tone is thus generated;

FIG. 22 is schematic diagrams illustrating radiation ranges of ultrasonic waves generated by an ultrasonic treatment apparatus according to the first modification of the fourth embodiment of the present invention and also illustrating a region where the radiation ranges are superposed on each other and a difference tone is thus generated;

FIG. 23A is a plane view showing an example of a focal interdigital transducer constituting an ultrasonic emitter according to a fifth embodiment of the present invention;

FIG. 23B is a sectional view showing an example of the focal interdigital transducer constituting the ultrasonic emitter according to the fifth embodiment of the present invention;

FIG. 24 is a schematic diagram illustrating the position of the focus of ultrasonic waves generated by an ultrasonic treatment apparatus according to the fifth embodiment of the present invention;

FIG. 25 is a schematic diagram illustrating the focal positions of SAWs and BAWs generated by the ultrasonic treatment apparatus according to the fifth embodiment of the present invention;

FIG. 26 is a plane view showing an example of an interdigital transducer constituting an ultrasonic emitter according to a first modification of the fifth embodiment of the present invention;

FIG. 27 is a plane view showing an example of a focal interdigital transducer constituting an ultrasonic emitter according to a seventh embodiment of the present invention;

FIG. 28 is a schematic diagram illustrating the position of the focus of ultrasonic waves generated by an ultrasonic treatment apparatus according to the seventh embodiment of the present invention;

FIG. 29 is a plane view showing another example of a focal interdigital transducer constituting an ultrasonic emitter according to a seventh embodiment of the present invention;

FIG. 30 is a sectional view showing an example of an ultrasonic element constituting an ultrasonic emitter according to an eighth embodiment of the present invention;

FIGS. 31A and 31B are sectional views showing other examples of the ultrasonic element constituting the ultrasonic emitter according to the eighth embodiment of the present invention;

FIG. 32A is a sectional view showing an example of an ultrasonic element constituting an ultrasonic emitter according to a tenth embodiment of the present invention;

FIG. 32B is a plane view showing an example of the ultrasonic element constituting the ultrasonic emitter according to the tenth embodiment of the present invention;

FIG. 33A is a sectional view showing another example of the ultrasonic element constituting the ultrasonic emitter according to the tenth embodiment of the present invention;

FIG. 33B is a plane view showing another example of the ultrasonic element constituting the ultrasonic emitter according to the tenth embodiment of the present invention; and

FIGS. 34A and 34B are sectional views showing other examples of the ultrasonic element constituting the ultrasonic emitter according to the tenth embodiment of the present invention.

### DETAILED DESCRIPTION OF THE INVENTION

### First Embodiment

A first embodiment of the present invention is described with reference to the drawings. An ultrasonic treatment apparatus according to the present embodiment is used for a treatment, for example, by destroying cells or by heating and coagulating a tissue. To this end, the ultrasonic treatment apparatus applies ultrasonic waves having a desired frequency to a target position. For example, to destroy cells, ultrasonic waves are directly applied to the cells so that the cells can be destroyed by the energy of the ultrasonic waves. Otherwise, microbubbles used as, for example, an ultrasonic contrast agent are provided to a part to be irradiated with ultrasonic waves, and the microbubbles are burst by ultrasonic irradiation. Thus, the cells can be destroyed by cavitation energy generated at the burst of the microbubbles.

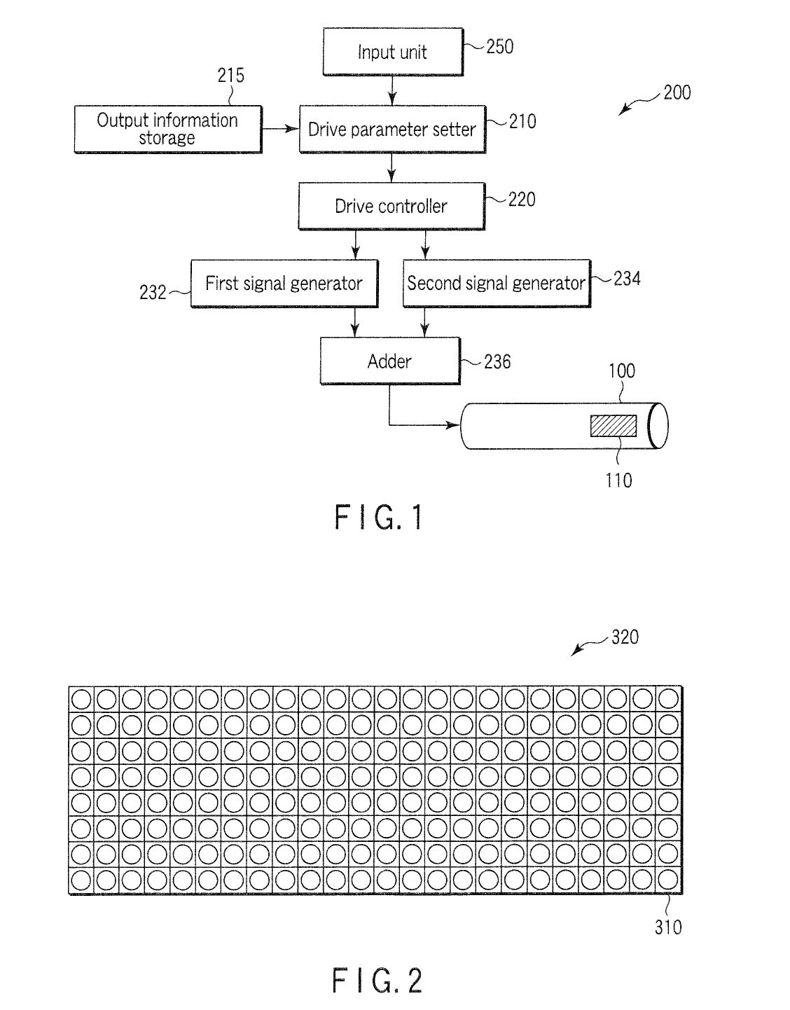

A rough configuration of the ultrasonic treatment apparatus according to the present embodiment is shown in FIG. 1. This ultrasonic treatment apparatus has a tip portion 100, an ultrasonic treatment apparatus controller 200 and an input unit 250. The tip portion 100 is, for example, cylindrically shaped. An ultrasonic wave emitter 110 which is a sound source of ultrasonic waves emitted by the ultrasonic treatment apparatus is disposed in a part of the circumferential surface of the tip portion 100. The ultrasonic treatment apparatus controller 200 has a drive parameter setter 210, an output information storage 215, a drive controller 220, a first signal generator 232, a second signal generator 234 and an adder 236.

The input unit 250 receives an operator instruction on, for example, an ultrasonic wave irradiation target position and the strength of ultrasonic waves to be radiated. The input unit 250 outputs the input operator instruction to the drive parameter setter 210. On the basis of the information input from the input unit 250, the drive parameter setter 210 reads frequency information from the output information storage 215. On the basis of the frequency information, the drive parameter setter 210 determines the frequency, amplitude and initial phase of the ultrasonic waves to be emitted from the ultrasonic wave emitter 110. The drive parameter setter 210 outputs the determined frequency, amplitude and initial phase of the ultrasonic waves to the drive controller 220.

The frequency information described later and the like are stored in the output information storage 215. At the request of the drive parameter setter 210, the output information storage 215 outputs the stored frequency information to the drive parameter setter 210. On the basis of the frequency, amplitude and initial phase of the ultrasonic waves input from the drive parameter setter 210, the drive controller 220 outputs an instruction to the first signal generator 232 and the second signal generator 234 to generate signals corresponding to the frequency, amplitude and initial phase of the ultrasonic waves.

On the basis of the instruction input from the drive controller 220 to generate ultrasonic signals, the first signal generator 232 and the second signal generator 234 respectively generate signals and output the signals to the adder 236. The adder 236 adds together the signals input from the first signal generator 232 and the second signal generator 234, and outputs a resultant drive signal to the ultrasonic wave emitter 110. The adder 236 may be, for example, a general adding circuit using an operational amplifier. The signals having different frequencies can be superposed by the adder 236, and the sound source can be driven by the resultant drive signals.

For example, the ultrasonic wave emitter 110 functions as a sound source for emitting finite amplitude acoustic waves including a first signal frequency and a second signal frequency. For example, the drive parameter setter 210 functions as a frequency setter for setting the first signal frequency and the second signal frequency in accordance with a target position of a treatment object with respect to the sound source. For example, the first signal generator 232, the second signal generator 234 and the adder 236 function as drive signal generators configured to generate a drive signal and to drive the sound source, the drive signal being adapted to emit, to the sound source. For example, the output information storage 215 functions as a frequency information storage for storing information that associates the target position with the first signal frequency and the second signal frequency. For example, the input unit 250 functions as a position information input unit for inputting the target position. For example, the first signal generator 232 and the second signal generator 234 function as signal generators which generate a first drive signal having the first signal frequency and a second drive signal having the second signal frequency. For example, the adder 236 functions as an adder which adds the first drive signal and the second drive signal together to generate an additional drive signal.

The ultrasonic wave emitter 110 according to the present embodiment is described. The ultrasonic wave emitter 110 according to the present embodiment is, as shown in its plane view of FIG. 2, a cMUT array 320 in which capacitive micromachined ultrasonic transducers (cMUTs) 310 are arrayed.

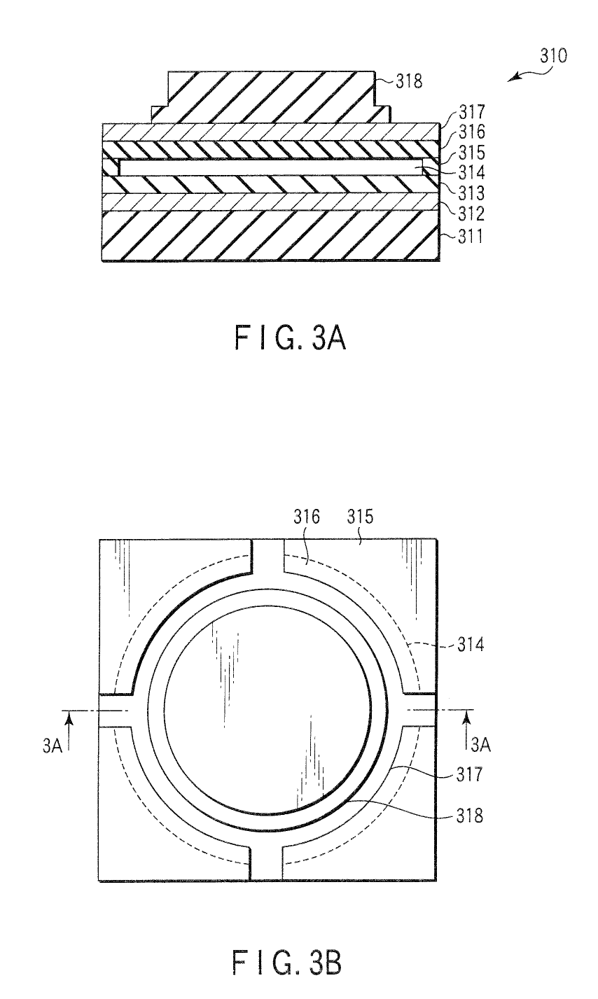

One cMUT 310 has, for example, a structure shown in a sectional view of FIG. 3A and in a plane view of FIG. 3B. The cMUT 310 is formed on a bottom substrate 311 made of, for example, silicon. A bottom electrode 312 made of, for example, Pt/Ti is formed on the bottom substrate 311. The material of the bottom electrode 312 is not exclusively Pt/Ti and may be, for example, Au/Cr, Mo, W, phosphor bronze or Al. A dielectric film 313 made of, for example, SrTiO₃ is formed on the bottom electrode 312. The material of the dielectric film 313 is not exclusively SrTiO₃ and can be a material having a high dielectric constant such as BaTiO₃, barium strontium titanate, tantalum pentoxide, niobia-stabilized tantalum pentoxide, aluminum oxide or TiO₂. This dielectric film 313 functions to increase a capacitance between a top electrode 317 and the bottom electrode 312 across a gap 314 described later.

A membrane supporter 315 made of, for example, SiN is present on the dielectric film 313 to have the cylindrical gap 314. A membrane 316 made of, for example, SiN is formed over the gap 314 and the membrane supporter 315. The top electrode 317 made of, for example, Pt/Ti as the bottom electrode 312 is formed on the membrane 316.

A convex head 318 having a smaller diameter than the gap 314 is provided on the top electrode 317. The head 318 is made of, for example, tetraethoxysilane (TEOS). The head 318 is thicker than the membrane 316 as a result of a semiconductor process. The material of the head 318 is not exclusively TEOS and can be some other material often used in the semiconductor process, such as SiN, SiO₂ or polyimide. Moreover, the head 318 may be a multilayer film of multiple materials.

In the cMUT 310 having such a configuration, when a voltage is applied across the top electrode 317 and the bottom electrode 312, an attractive force acts between these electrodes. When the application of the voltage is stopped, an original state is restored. The membrane 316 and the head 318 formed thereon are vibrated by periodical voltage applications. As a result, ultrasonic waves having a finite amplitude are radiated from the cMUT 310. By varying the frequency of the voltage applied across the top electrode 317 and the bottom electrode 312, the cMUT 310 can output ultrasonic waves having various frequencies.

In the cMUT 310, the part of the membrane 316 that lies over the gap 314 is relatively soft, and the top electrode 317 and the head 318 are relatively rigid. Therefore, when the membrane 316, the top electrode 317 and the head 318 are taken as a whole, the place where the head 318 is located is relatively rigid, and the part therearound is relatively soft. Thus, the direction of the vibration and displacement of the membrane 316 during the application of the voltage across the bottom electrode 312 and the top electrode 317 is unified to the direction of a normal to the top surface of the convex portion of the head 318. That is, while the membrane of the cMUT which does not include the head 318 vibrates in a bending manner, the membrane 316 of the cMUT 310 which includes the head 318 makes one-direction vibrations similar to thickness longitudinal vibrations. That is, the part of the top electrode 317 and the head 318 thereon can make vibrations similar to piston vibrations. As a result, according to this cMUT 310, the directivity of emitted ultrasonic waves increases, and a high sound pressure effect can be obtained at the target position. Moreover, such a configuration can reduce unnecessary vibrations such as harmonic vibrations generated due to bending vibration of the membrane 316.

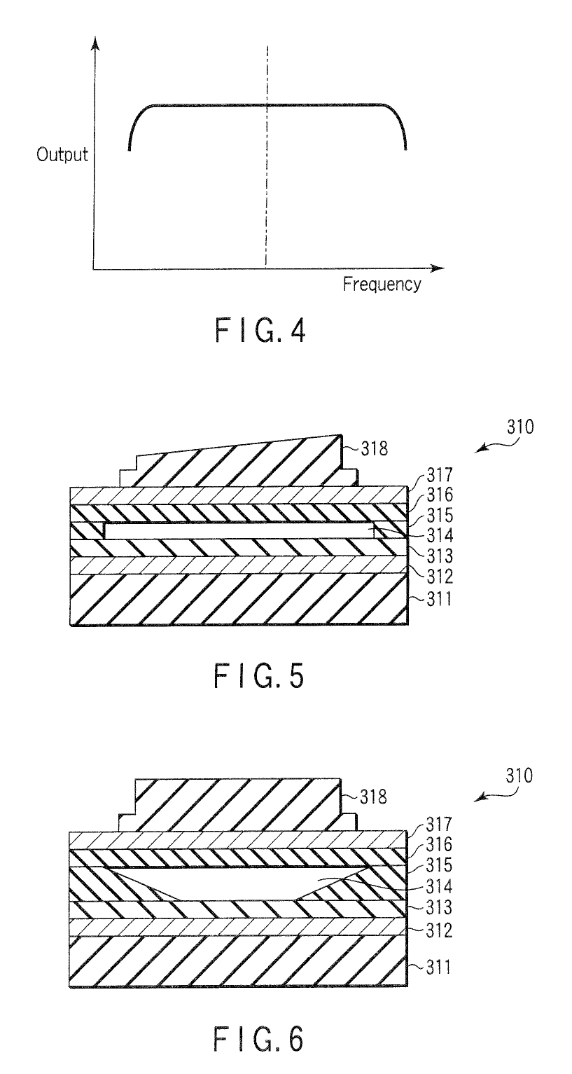

The frequency characteristics of the cMUT 310 having the configuration described above are shown in FIG. 4. As shown in the graph, the cMUT 310 has a stable output over a wide frequency range around a center frequency indicated by a dashed-dotted line. The center frequency ranges is designed to from, for example, more than ten MHz to several ten MHz, and its band is designed to be, for example, 50 to 100%. The cMUT 310 is also characterized by reduced device-to-device variation owing to the stabilization of its manufacturing process.

As shown in FIG. 5, the head 318 may be sloped. When the head 318 is thus sloped, ultrasonic waves are emitted in the direction of a normal to the slope, i.e., in a direction diagonal to the bottom substrate 311.

The shape of the gap 314 in the cMUT 310 may be modified as shown in FIG. 6. That is, the cross-sectional areas of the surfaces parallel to the surface of the bottom substrate 311 on the bottom electrode 312 side and the top electrode 317 side may be different. Such a shape enables the band of output ultrasonic waves to be broader than when the gap 314 has a cylindrical shape as in the cMUT shown in FIG. 3.

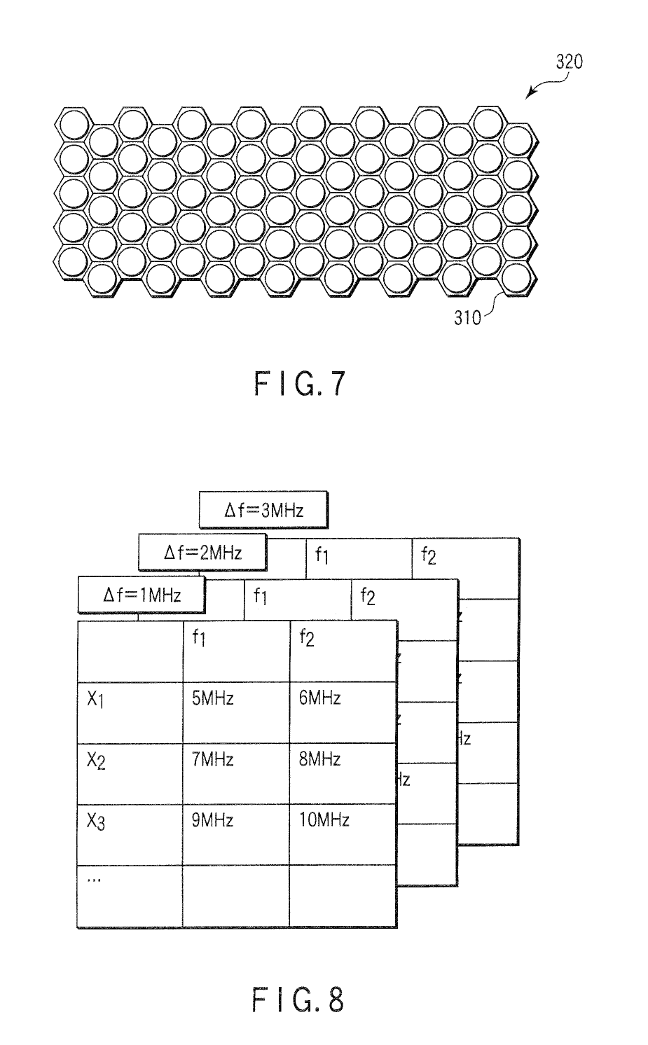

The cMUT 310 may have some other polygonal planar shape such as a hexagonal planar shape shown in FIG. 7 instead of the above-mentioned quadrangular planar shape, and such cMUTs 310 may be arrayed to form the cMUT array 320. The planar shape of the head 318 is not exclusively circular either.

The cMUT array 320 is an array of a great number of cMUTs 310, and the bottom electrodes 312 of the respective cMUTs 310 are electrically connected to one another. The top electrodes 317 are also connected to one another. Thus, all of the cMUTs 310 vibrate at the same time. The ultrasonic wave emitter 110 having the cMUT array 320 comprising such cMUTs 310 is driven by the drive signal input from the adder 236 and outputs ultrasonic waves.

The radiation of ultrasonic waves to a target part by the ultrasonic treatment apparatus according to the present embodiment is described. One use of the ultrasonic treatment apparatus is, for example, to destroy cells by ultrasonic irradiation. For example, it is known that if microbubbles are provided to a part in which cells should be destroyed and the microbubbles are burst by ultrasonic irradiation, surrounding cells can be efficiently destroyed by cavitation energy generated by the burst of the microbubbles. In bursting the microbubbles, the efficiency of bursting the microbubbles is improved if the frequency of ultrasonic waves to be radiated is set at a value close to the resonance frequency of the microbubbles. For example, when a commercially available ultrasonic contrast agent is used as the microbubbles, the frequency of ultrasonic waves is appropriately about 1 to 2 MHz.

In general, ultrasonic waves of a higher frequency propagating through a substance are damped more. When the cMUT 310 is used as described above, the frequency of ultrasonic waves to be emitted can be changed. Thus, when an optimum frequency is selected in accordance with the depth of the target position to be irradiated with ultrasonic waves, the ultrasonic waves can be efficiently propagated to the target position.

In view of the circumstances, the frequency of ultrasonic waves to be emitted is considered as below in the ultrasonic treatment apparatus according to the present embodiment. For example, the frequency of the signal generated by the first signal generator 232 is f₁, the angular frequency thereof is ω₁, and the amplitude thereof is A. The frequency of the signal generated by the second signal generator 234 is f₂, the angular frequency thereof is ω₂, and the amplitude thereof is A. Here, the relation between f₁ and ω₁ and the relation between f₂ and ω₂ are as below.

ω₁=2PIf₁

ω₂=2PIf₂

The signal generated by the first signal generator 232 and the signal generated by the second signal generator 234 are input to the adder 236, and the adder 236 adds these signals together. In this example, if the initial phase of the signal generated by the first signal generator 232 is coincident with the initial phase of the signal generated by the second signal generator 234, an additional signal x(t) generated by the adder 236 is represented by Equation (1).

$$\begin{matrix}x ( t ) = A cos ( {ω}_{1} t ) + A cos ( {ω}_{2} t ) \\ = 2 A cos \frac{{ω}_{1} + {ω}_{2}}{2} t cos \frac{{ω}_{1} - {ω}_{2}}{2} t\end{matrix} \qquad ( 1 )$$

Here, x(t)=2A cos(((ω₁+ω₂)/2)t) is referred to as carrier waves, and x(t)=cos(((ω₁−ω₂)t) is referred to as modulated waves. When the ultrasonic waves generated by the ultrasonic wave emitter 110 are applied to a subject in accordance with the additional signal x(t) represented by Equation (1), the ultrasonic waves having the frequency of the modulated waves are propagated by the ultrasonic waves having the frequency of the carrier waves. Thus, in order to apply ultrasonic waves of 1 to 2 MHz to the target position to burst the microbubbles as described above, f₁ and f₂ can be determined so that the frequency Δf=|f₁−f₂|=(|ω₁−ω₂|)/2PI of the modulated waves may be 1 to 2 MHz.

As described above, ultrasonic waves of a higher frequency propagating through a substance are damped more. Thus, when the target position is far (deep) from the ultrasonic wave emitter 110 which is the source of the ultrasonic waves, f₁ and f₂ can be determined so that the frequency (f₁+f₂)=(ω₁+ω₂)/2PI of the carrier waves may be low. When the target position is close (shallow) to the ultrasonic wave emitter 110, f₁ and f₂ can be determined so that the frequency (f₁+f₂)=(ω₁+ω₂)/2PI of the carrier waves may be high. The frequency of the carrier waves is desirably determined on the basis of the distance from the ultrasonic wave emitter 110 to the target position and on the basis of, for example, the ultrasonic wave absorption coefficient of the substance present between the ultrasonic wave emitter 110 and the target position.

For example, ultrasonic waves generated by the ultrasonic wave emitter 110 in accordance with the additional signal x(t) represented by Equation (1) are radiated. In this case, owing to the nonlinearity of a medium that conveys acoustic waves, an effect equivalent to generating the modulated waves having the frequency Δf=|f₁−f₂| at the target position can be obtained. Such an effect is referred to as a self-demodulation effect. This self-demodulation effect is one of the important points of the present embodiment. If f₁ and f₂ that simultaneously satisfy the frequency of the modulated waves and the frequency of the carrier waves are determined, the ultrasonic treatment apparatus can apply ultrasonic waves having a desired frequency to the target position regardless of the difference of the depth of the target position. In the present embodiment, the drive parameter setter 210 determines the values of f₁ and f₂.

Equation (1) used in the above explanation is one example. It is possible to use other additional signals which are generated by using the signals output from the first signal generator 232 and the second signal generator 234 and which are the product of the carrier waves and the modulated waves. For example, an amplitude A₁ of the signal generated by the first signal generator 232 may be different from an amplitude A₂ of the signal generated by the second signal generator 234. Moreover, there may be a difference between the initial phases of the signals generated by the first signal generator 232 and the second signal generator 234.

Now, the operation of the ultrasonic treatment apparatus according to the present embodiment is described. When this ultrasonic treatment apparatus is used, the ultrasonic wave emitter 110 of the tip portion 100 is brought into contact with a subject (sound propagating medium) to be irradiated with ultrasonic waves. There may be an acoustic matching layer for matching acoustic impedance between the ultrasonic wave emitter 110 and the subject, or the ultrasonic wave emitter 110 and the subject may be in direct contact. As the acoustic matching layer, it is possible to use, for example, a water bag containing deaerated water, or a sound matching material made of a resin or jellylike substance generally used in an ultrasonic diagnostic apparatus or ultrasonic treatment apparatus.

The ultrasonic treatment apparatus generates ultrasonic waves in accordance with an operator instruction input from the input unit 250 while the ultrasonic wave emitter 110 is in contact with the subject. The operator can use the input unit 250 to designate an ultrasonic wave irradiation target position. In this case, the drive parameter setter 210 of the ultrasonic treatment apparatus controller 200 first determines frequencies of the carrier waves and the modulated waves as described above in accordance with a target position and an object input from the input unit 250 and designated by the operator. The drive parameter setter 210 then determines the frequency f₁, amplitude A₁ and initial phase θ₁ of the signal generated by the first signal generator 232 and the frequency f₂, amplitude A₂ and initial phase θ₂ of the signal generated by the second signal generator 234 to correspond to the determined carrier waves and modulated waves. Here, the drive parameter setter 210 uses the frequency information stored in the output information storage 215.

Tables which are prepared for Δf=|f₁−f₂| and which contain information on combinations of f₁ and f₂ corresponding to depths X of the target position, for example, as schematically shown in FIG. 8 are stored in the output information storage 215. The drive parameter setter 210 reads a necessary table from the output information storage 215 in accordance with the target position and object, and determines a combination of f₁ and f₂ on the basis of this table. It should be understood that the tables shown in FIG. 8 are illustrative only and any table that indicates a combination of f₁ and f₂ corresponding to the depth X of the target position may be used. Moreover, some or all of the relations between X, f₁ and f₂ may be represented by functions, and the drive parameter setter 210 may perform a calculation on the basis of the functions.

The combinations of f₁ and f₂ corresponding to the depth X of the target position are previously stored in the output information storage 215, so that the drive parameter setter 210 can rapidly determine a combination of f₁ and f₂ in accordance with the target position and object.

Tables concerning the amplitude A₁ and initial phase θ₁ of the ultrasonic waves generated by the first signal generator 232 and the amplitude A₂ and initial phase θ₂ of the ultrasonic waves generated by the second signal generator 234 that are adapted to use are also stored in the output information storage 215.

The drive parameter setter 210 outputs the determined f₁, f₂, A₁, A₂, θ₁ and θ₂ to the drive controller 220. The drive controller 220 instructs the first signal generator 232 to output a signal of the frequency f₁, amplitude A₁ and initial phase θ₁, on the basis of the value input from the drive parameter setter 210. The drive controller 220 also instructs the second signal generator 234 to output a signal of the frequency f₂, amplitude A₂ and initial phase θ₂.

The first signal generator 232 and the second signal generator 234 respectively generate signals on the basis of the instructions input from the drive controller 220 to generate ultrasonic waves. The first signal generator 232 and the second signal generator 234 respectively output the generated signals to the adder 236. The adder 236 adds together the signals input from the first signal generator 232 and the second signal generator 234, and outputs, to the ultrasonic wave emitter 110, a drive signal which is the additional signal represented by, for example, Equation (1).

Each of the cMUTs 310 constituting the cMUT array 320 of the ultrasonic wave emitter 110 vibrates the head 318 to emit ultrasonic waves in response to the drive signal input from the adder 236. As a result, the ultrasonic waves having the frequency Δf=|f₁−f₂| to be self-demodulated at the target position as described above propagate through the subject.

The following advantages are obtained by the ultrasonic treatment apparatus according to the present embodiment. Even if the ultrasonic wave emitter 110 is used which has to have a high frequency of the ultrasonic waves to be output, for example, a center frequency ranging from more than ten MHz to several ten MHz due to the characteristics of the cMUT and element size reduction, it is possible to radiate ultrasonic waves of, for example, 1 to 2 MHz which is the resonance frequency of the microbubbles. This ultrasonic treatment apparatus can radiate ultrasonic waves having a frequency proper for the object regardless of the configuration of the ultrasonic wave emitter 110. A frequency of the carrier waves is properly selected in such a manner as to take the damping of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use of the high-frequency ultrasonic waves advantageously allows for a sharper beam pattern.

### Second Embodiment

A second embodiment of the present invention is described with reference to the drawings. Here, the difference between the second embodiment and the first embodiment is described, and the same parts as those in the first embodiment are provided with the same reference numbers and are not described. The present embodiment concerns an endoscopic ultrasonic diagnostic treatment apparatus that has the ultrasonic treatment apparatus according to the first embodiment.

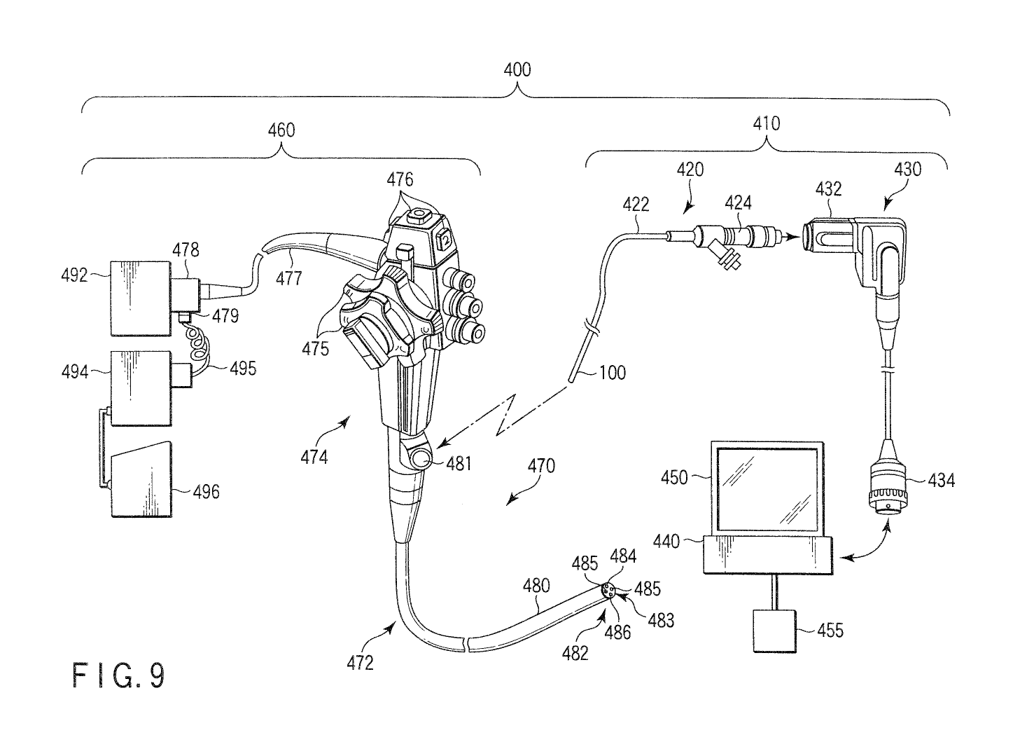

An endoscopic ultrasonic diagnostic treatment apparatus 400 according to the present embodiment mainly comprises an ultrasonic device 410 and an endoscopic device 460, as shown in FIG. 9. The ultrasonic device 410 has an ultrasonic probe 420 equipped with an elongate probe insertion tube 422. As shown in FIG. 9, the ultrasonic probe 420 comprises the tip portion 100 of the ultrasonic treatment apparatus according to the first embodiment. The ultrasonic probe 420 is inserted into a body cavity, and emits ultrasonic waves from the ultrasonic wave emitter 110 which is disposed in the tip portion 100 and which is not shown in FIG. 9. The ultrasonic probe 420 ultrasonically diagnoses and ultrasonically treats a desired living tissue in the body cavity by the emission of the ultrasonic waves. As has been described in the first embodiment, the ultrasonic wave emitter 110 performs a treatment to radiate ultrasonic waves to a target position and destroy cells at the target position by the energy of the ultrasonic waves. In addition, the ultrasonic device 410 has a function of ultrasonically diagnosing, before, after and during a treatment operation, a living tissue located in the vicinity of the target position to be treated.

The ultrasonic probe 420 is connected to an ultrasonic wave control unit 440 via a probe connecting unit 430. The ultrasonic probe 420 and the probe connecting unit 430 are removably connected to each other by a connecting portion 424 provided at the proximal end of the ultrasonic probe 420 and by a coupling portion 432 of the probe connecting unit 430. The probe connecting unit 430 and the ultrasonic wave control unit 440 are connected to each other by a connector 434 of the probe connecting unit 430. The ultrasonic wave control unit 440 is a part which includes the ultrasonic treatment apparatus controller 200 in the first embodiment and which controls the ultrasonic probe 420.

The ultrasonic wave control unit 440 generates a video signal on the basis of the signal obtained by the ultrasonic probe 420. The ultrasonic device 410 has an ultrasonic image display unit 450 for displaying an ultrasonic tomogram in accordance with the video signal generated by the ultrasonic wave control unit 440. The ultrasonic device 410 also has an input unit 455 for receiving operator inputs.

The endoscopic device 460 has an electronic endoscope 470 including an imaging device therein, a light source unit 492 for supplying an illumination light flux to the electronic endoscope 470, a video processor 494 and an endoscopic image display unit 496. The video processor 494 drives the unshown imaging device in the electronic endoscope 470, and receives an electric signal transmitted from the imaging device and processes the signal in various forms to generate a video signal for displaying an endoscopic observation image. The video signal generated by the video processor 494 is input to the endoscopic image display unit 496, and the endoscopic image display unit 496 displays the endoscopic observation image.

The electronic endoscope 470 mainly comprises an elongate insertion portion 472 to be inserted into the body cavity, an operation portion 474 disposed on the proximal side of the insertion portion 472, and a universal cord 477 extending from the side part of the operation portion 474.

An endoscope connector 478 to be connected to the light source unit 492 is provided at the end of the universal cord 477. An electric connector 479 is provided on the side part of the endoscope connector 478. A video cable 495 extending from the video processor 494 is connected to the electric connector 479.

A curving portion 480 is provided on the tip side of the insertion portion 472, and a hard portion 482 is further provided on the tip side of the curving portion 480. A tip face 483 of the hard portion 482 is provided with an illumination light window 484 and an observation window 485 for direct-vision endoscopic observation, and a forceps exit 486.

The operation portion 474 is provided with an angle knob 475, operation switches 476, and a treatment tool insertion port 481. The angle knob 475 is used to control the curving operation of the curving portion 480 of the insertion portion 472. The operation switches 476 are used to change display images displayed on a display screen of the endoscopic image display unit 496 and to indicate various operations such as a freeze operation or release operation. The treatment tool insertion port 481 is an introduction opening for, for example, a treatment tool to be introduced into the body cavity, and is in communication with the above-mentioned forceps exit 486.

The ultrasonic wave control unit 440 is described. By combined with the endoscopic device 460, the ultrasonic device 410 according to the present embodiment can be used for the treatment of irradiating the target position with ultrasonic waves in the body cavity as described in the first embodiment. Moreover, when the cMUT array 320 of the ultrasonic wave emitter 110 is used to emit and receive ultrasonic waves, the ultrasonic wave control unit 440 can also function as an ultrasonic image diagnostic apparatus.

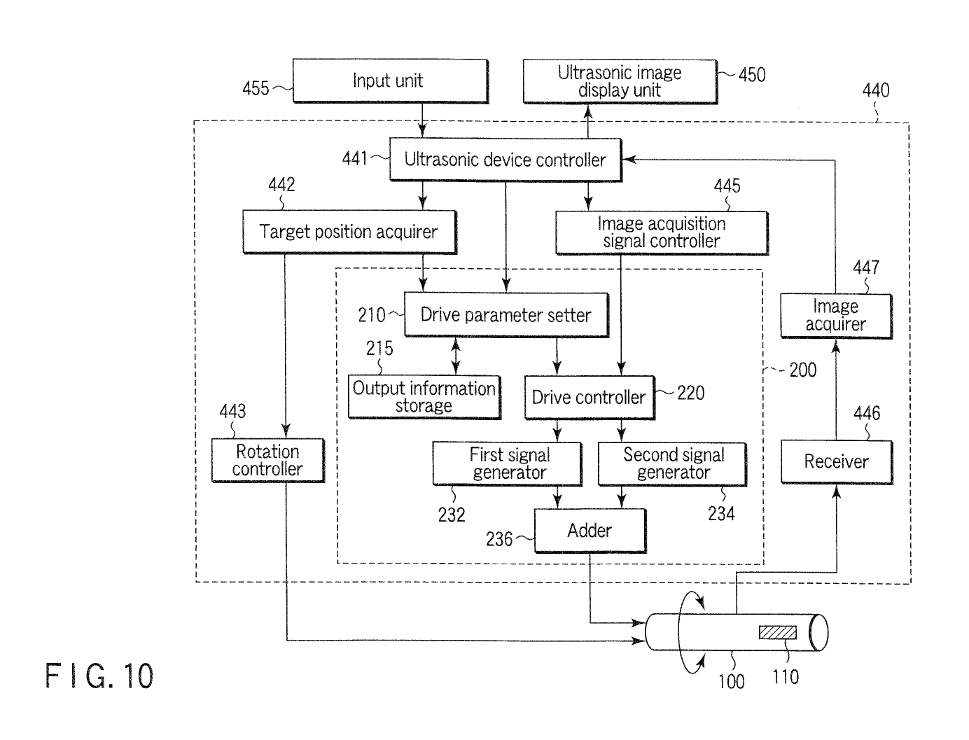

As shown in FIG. 10, the ultrasonic wave control unit 440 which controls the ultrasonic device 410 has the ultrasonic treatment apparatus controller 200 described in the first embodiment. The ultrasonic wave control unit 440 also has an ultrasonic device controller 441 for controlling the whole ultrasonic wave control unit 440, a target position acquirer 442, a rotation controller 443, an image acquisition signal controller 445, a receiver 446 and an image acquirer 447.

The tip portion 100 can be rotated by an unshown rotation device to enable image acquisition and ultrasonic wave radiation in various directions within the body cavity. In order to control this rotation, the target position acquirer 442 acquires a direction to radiate ultrasonic waves from the ultrasonic device controller 441. The target position acquirer 442 outputs, to the rotation controller 443, information on the direction to radiate ultrasonic waves. The rotation controller 443 controls the operation of the unshown rotation device in accordance with the ultrasonic wave radiation direction input from the target position acquirer 442. Further, the target position acquirer 442 acquires, from the ultrasonic device controller 441, an ultrasonic wave irradiation target position for an ultrasonic treatment, and outputs the target position to the drive parameter setter 210. The drive parameter setter 210 determines a frequency of ultrasonic waves to be radiated in accordance with the target position input from the target position acquirer 442, as described in the first embodiment.

An ultrasonic image diagnosis is carried out in a generally known manner. Thus, the image acquisition signal controller 445 determines various parameters of an ultrasonic pulse suitable for the image diagnosis. The image acquisition signal controller 445 also controls the drive controller 220. The image acquisition signal controller 445 outputs the determined value to the drive controller 220. When ultrasonic waves having a pure sound are used, the drive controller 220 only uses the first signal generator 232. The drive controller 220 outputs a signal generation instruction to the first signal generator 232 as in the first embodiment.

In the ultrasonic image diagnosis, each of the cMUTs 310 of the ultrasonic wave emitter 110 serves as a receiving element. The receiver 446 acquires the signal received by the cMUTs 310. The receiver 446 outputs the acquired signal to the image acquirer 447. The image acquirer 447 constructs an ultrasonic image in a generally known manner on the basis of the signal input from the receiver 446. The image acquirer 447 outputs the constructed image to the ultrasonic device controller 441.

The ultrasonic device controller 441 performs various calculations and controls the respective parts. The ultrasonic device controller 441 also receives an input from the input unit 455. Moreover, the ultrasonic device controller 441 outputs the ultrasonic image input from the image acquirer 447 to the ultrasonic image display unit 450. For example, the image acquisition signal controller 445 functions as an image acquisition signal setter which sets an image acquisition emission ultrasonic wave signal.

The operation of the endoscopic ultrasonic diagnostic treatment apparatus 400 according to the present embodiment is described. The endoscopic device 460 is a generally known endoscopic device and is not directly related to the present invention, and is therefore not described. The operator inserts the insertion portion 472 into the body cavity of the subject in a treatment, and brings the end of the insertion portion 472 to a position of the subject to be diagnosed or treated. In the treatment, microbubbles are administered.

The probe insertion tube 422 is inserted from the treatment tool insertion port 481 of the endoscopic device 460 and passed through the insertion portion 472, and the tip portion 100 extends from the forceps exit 486. At a position where a diagnosis or treatment is to be conducted, the ultrasonic device controller 441 instructs the respective parts to perform the ultrasonic image diagnosis.

The ultrasonic device controller 441 outputs, to the target position acquirer 442, an angle at which an image is to be acquired. The target position acquirer 442 outputs, to the rotation controller 443, the image acquisition angle input from the ultrasonic device controller 441. The rotation controller 443 controls the unshown rotation device in accordance with the value input from the target position acquirer 442. For example, when the acquisition of an image around the whole circumference of the tip portion 100 is requested, the rotation controller 443 controls the rotation device to, for example, continuously rotate the tip portion 100 at a regular velocity in consideration of the time required for the image acquisition. When the acquisition of an image, for example, within a certain angle of circumference rather than around the whole circumference of the tip portion 100 is requested, the rotation controller 443 may rotate the tip portion 100 repeatedly back and forth.

The ultrasonic device 410 acquires an ultrasonic image while rotating the tip portion 100 as described above. In accordance with the instruction from the ultrasonic device controller 441, the image acquisition signal controller 445 controls the output of ultrasonic waves for acquiring an ultrasonic image. For example, the output ultrasonic waves are high-frequency pulse waves having a frequency of about 5 to 20 MHz and a pulse width of about several μ seconds. The image acquisition signal controller 445 outputs such information to the drive controller 220.

In acquiring the ultrasonic image, the first signal generator 232 is only used for signal generation because the generated ultrasonic waves have a pure tone. The drive controller 220 outputs an instruction for the first signal generator 232 to generate a signal on the basis of the value input from the image acquisition signal controller 445. The first signal generator 232 generates a signal in response to the instruction from the drive controller 220, and outputs the signal to the adder 236. The signal to be input to the adder 236 is the signal generated by the first signal generator 232 alone, so that the adder 236 outputs the signal input from the first signal generator 232 directly to the ultrasonic wave emitter 110. In response to the signal input from the adder 236, the ultrasonic wave emitter 110 vibrates the head 318 of the cMUT 310 to emit ultrasonic waves.

The ultrasonic waves emitted from the ultrasonic wave emitter 110 propagate through the subject. The propagated ultrasonic waves are reflected in accordance with the acoustic characteristics of the subject. The cMUT 310 of the ultrasonic wave emitter 110 captures the reflected waves by the head 318. That is, the cMUT 310 is vibrated by the reflected waves. As a result, a voltage between the top electrode 317 and the bottom electrode 312 of the cMUT 310 is changed. The ultrasonic wave emitter 110 outputs this electric signal to the receiver 446.

The receiver 446 acquires the electric signal from the ultrasonic wave emitter 110, and outputs the electric signal to the image acquirer 447. The image acquirer 447 constructs an image of the inside of the subject in a generally known manner on the basis of the signal input from the receiver 446. An example of the constructed image is schematically represented in FIG. 11. In the example of FIG. 11, the internal structure of the subject in the whole circumferential direction of the tip portion 100 is observed. Here, a center 810 cannot be imaged because of the presence of the tip portion 100. Moreover, an image acquirable range 820 exists depending on the range in which the emitted ultrasonic waves reach and the reflected waves can be detected. In this diagram, for example, the position of an object 830 can be indicated by a rotation angle θ based on an axis defined by the posture of the tip portion 100 and by a distance r from the center of the tip portion 100.

The image acquirer 447 outputs the acquired image to the ultrasonic device controller 441. The ultrasonic device controller 441 outputs the image input from the image acquirer 447 to the ultrasonic image display unit 450. The ultrasonic image display unit 450 displays, for example, an ultrasonic image shown in FIG. 11 input from the ultrasonic device controller 441.

The operator determines a target position to be treated while checking the image displayed on the ultrasonic image display unit 450. The input unit 455 receives the input of the treatment target position determined by the operator. The operator also determines a frequency of ultrasonic waves to be radiated, in accordance with the treatment target. The input unit 455 receives the frequency of the ultrasonic waves to be radiated to the treatment target determined by the operator. Here, general input means such as a keyboard, mouse or joystick may be used as the input unit 455.

The input unit 455 outputs the operator instruction to the ultrasonic device controller 441. In accordance with the operator instruction input from the input unit 455, the ultrasonic device controller 441 controls the ultrasonic wave emitter 110 to emit treatment ultrasonic waves. The ultrasonic device controller 441 outputs an instruction for the image acquisition signal controller 445 to stop the emission of the ultrasonic waves for acquiring an ultrasonic image. On the other hand, the ultrasonic device controller 441 outputs, to the target position acquirer 442, the target position input from the operator.

In accordance with the target position input from the ultrasonic device controller 441, the target position acquirer 442 outputs a desired rotation angle (value associated with θ in FIG. 11) of the tip portion 100 to the rotation controller 443 in order to direct the ultrasonic wave emitter 110 to the target position. On the basis of the input from the target position acquirer 442, the rotation controller 443 controls the rotation device, and rotates the tip portion 100 to direct the ultrasonic wave emitter 110 to the target position.

The target position acquirer 442 outputs, to the drive parameter setter 210, the distance (r in FIG. 11) of the target position from the ultrasonic wave emitter 110. The ultrasonic device controller 441 outputs, to the drive parameter setter 210, the frequency of the radiation ultrasonic waves input from the input unit 455. On the basis of the target position input from the target position acquirer 442 and the frequency of the radiation ultrasonic waves input from the ultrasonic device controller 441, the drive parameter setter 210 calculates the frequency of the ultrasonic waves to be output, as has been described in the first embodiment. The ultrasonic waves are then emitted as has been described in the first embodiment. As a result, the ultrasonic waves having the set frequency are propagated to the target position, and the desired ultrasonic waves are applied to the target position.

The tissue degenerated by the ultrasonic wave irradiation is lower in ultrasonic wave propagation efficiency than the tissue before ultrasonic wave irradiation. Therefore, it is preferable to radiate ultrasonic waves from far parts (the side far from the ultrasonic wave emitter 110) to near parts (the side close to the ultrasonic wave emitter 110) in order.

The endoscopic ultrasonic diagnostic treatment apparatus 400 may be configured so that the procedure of ultrasonic wave radiation from the far parts to the near parts may be performed regardless of the operator instruction. Control may be performed so that ultrasonic waves are radiated in order by a predetermined procedure within a desired ultrasonic wave radiation range designated by the operator. For such control, for example, the drive parameter setter 210 gradually changes the frequency of the carrier waves for the output ultrasonic waves in increments within the range designated by the operator.

The endoscopic ultrasonic diagnostic treatment apparatus 400 according to the present embodiment ensures that an image of the inside of the subject is acquired by the ultrasonic device 410, and at the same time, the ultrasonic waves having the frequency of the set modulated waves can be applied to various target positions. That is, the rotation angle of the tip portion 100 can be controlled in accordance with the target position to be irradiated with ultrasonic waves. Moreover, a frequency of the carrier waves is properly selected in such a manner as to take the damping of the ultrasonic waves into consideration, thereby ensuring that the ultrasonic waves having the set frequency can be applied to the target position. For example, microbubble cavitation can be highly efficiently generated, thereby ensuring that cells can be destroyed by low-energy ultrasonic waves.

Furthermore, according to the endoscopic ultrasonic diagnostic treatment apparatus 400 of the present embodiment, the tip portion 100 is driven as has been described in the first embodiment. Thus, the same ultrasonic wave generating element can be used to generate ultrasonic waves having an image acquisition frequency of about 5 to 20 MHz and ultrasonic waves of, for example, 1 to 2 MHz which is the resonance frequency of the microbubbles.

### First Modification of Second Embodiment

A first modification of the second embodiment is described with reference to the drawings. Here, the difference between the present modification and the second embodiment is described, and the same parts as those in the second embodiment are provided with the same reference numbers and are not described. The tip portion 100 according to the second embodiment is configured so that the ultrasonic wave emitter 110 is circumferentially rotated by the rotation controller 443 and the unshown rotation device.

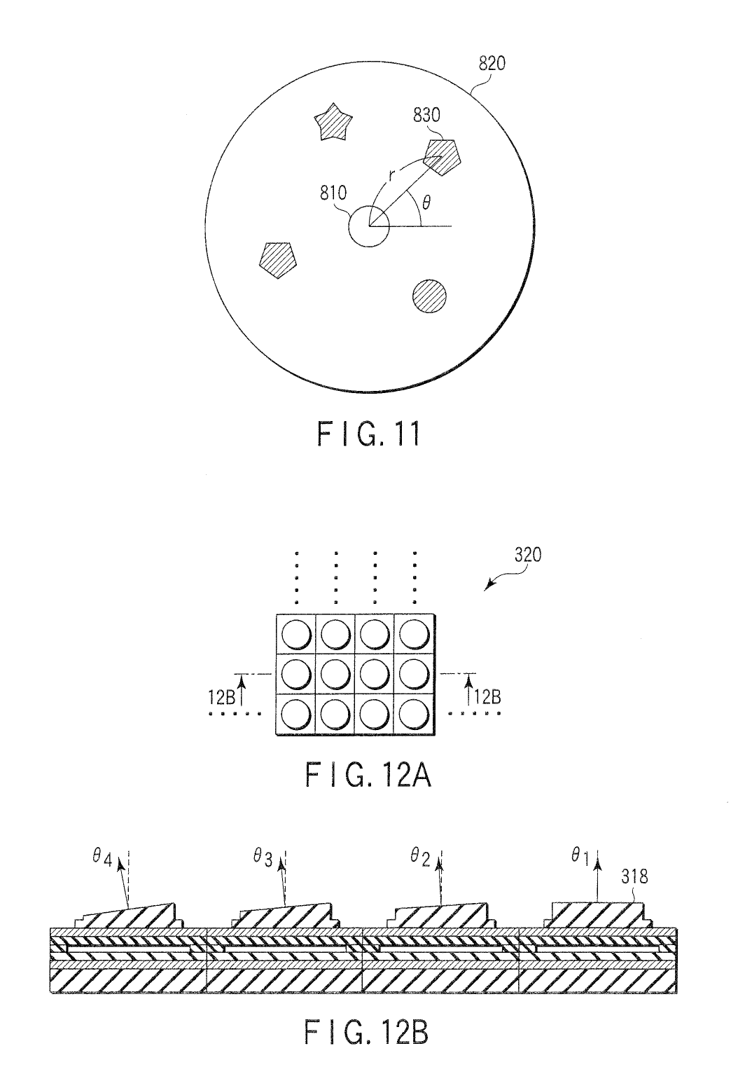

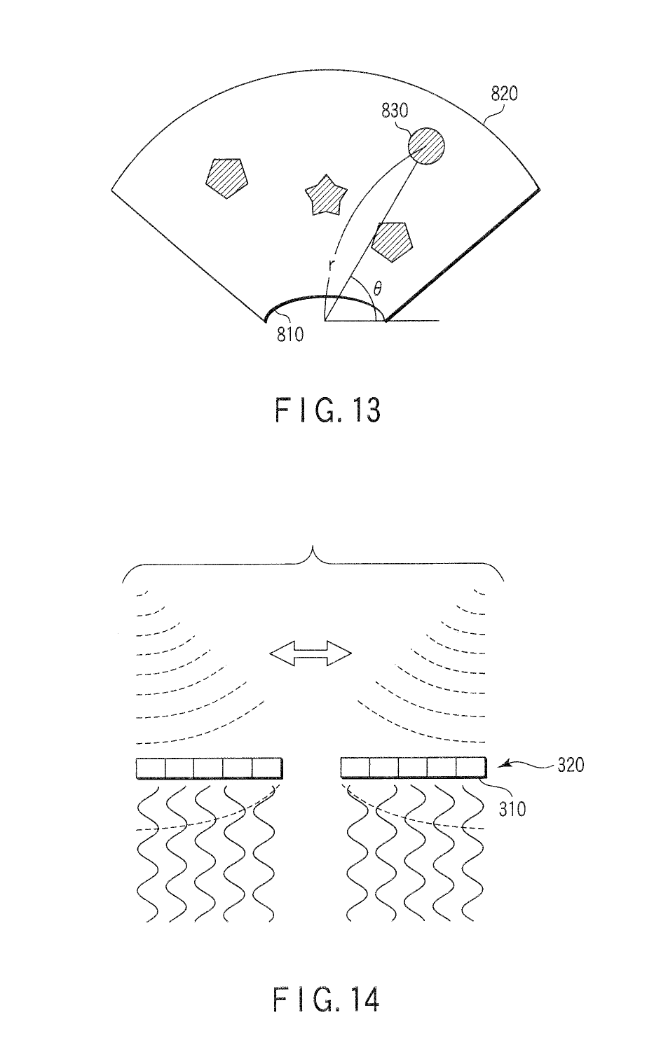

In contrast, according to this modification, an ultrasonic wave emitter 110 does not physically rotate but is configured to be able to change the radiation direction of ultrasonic waves. That is, an enlarged plane view of part of a cMUT array 320 is shown in FIG. 12A, and a sectional view through 12B-12B in FIG. 12A is shown in FIG. 12B. As shown in FIG. 12B, heads 318 of cMUTs 310 constituting the cMUT array 320 have different slopes depending on the position in the cMUT array 320. The heads 318 having the same slope are grouped together. The cMUT array 320 is configured to be able to control a voltage difference between a top electrode 317 and a bottom electrode 312 of the cMUT 310 on a group basis. Thus, the emission direction of ultrasonic waves can be changed by selecting a group of the cMUTs 310 to emit ultrasonic waves. Such a change of the emission direction of ultrasonic waves can also be applied to ultrasonic waves for acquiring an ultrasonic image and ultrasonic waves for treatment. A schematic diagram of an ultrasonic image obtained by the endoscopic ultrasonic diagnostic treatment apparatus 400 having such a configuration is shown in FIG. 13. There is no essential difference between the image obtained by this modification and the image obtained by the second embodiment.

According to the endoscopic ultrasonic diagnostic treatment apparatus 400 having such a configuration, advantages similar to the advantages of the endoscopic ultrasonic diagnostic treatment apparatus 400 according to the second embodiment can be obtained.

Furthermore, the endoscopic ultrasonic diagnostic treatment apparatus 400 may be configured to use the ultrasonic wave emitter 110 in which all of the heads 318 have the same slope as in the second embodiment so that the radiation direction of ultrasonic waves is changed by a phased array. The phased array is briefly described. The relation between the phase and wave front of emitted ultrasonic waves is shown in FIG. 14. In this diagram, a middle row in the right and left parts schematically shows the cMUT array 320. Here, squares indicate the cMUTs 310 that constitute the cMUT array 320. In the lower row of this diagram, the phases of the ultrasonic waves emitted from the cMUTs 310 are schematically shown by solid lines. In the upper row of this diagram, the wave fronts of the emitted ultrasonic waves are schematically shown by broken lines. As shown, the ultrasonic waves emitted from the respective cMUTs 310 gradually come out of phase in accordance with the positions of the cMUTs 310 in the cMUT array 320, so that the traveling directions of the emitted ultrasonic waves are different. In FIG. 14, the left side shows that the ultrasonic waves are traveling leftward in the diagram, and the right side shows that the ultrasonic waves are traveling rightward in the diagram.

In this modification, the adder 236 adds together signals input from a first signal generator 232 and a second signal generator 234, and then adds a phase difference to the additional signal in accordance with the positions of the cMUTs 310 in the cMUT array 320. Further, the adder 236 outputs, to the cMUTs 310 in the cMUT array 320, a drive signal which is the additional signal to which the phase difference is added. Thus, the radiation direction of emitted ultrasonic waves can also be changed by using the phased array.

Such a configuration can also provide advantages similar to the advantages of the endoscopic ultrasonic diagnostic treatment apparatus 400 according to the second embodiment. A tip portion 100 and ultrasonic treatment apparatus controller 200 according to this modification are not exclusively incorporated in the endoscopic ultrasonic diagnostic treatment apparatus 400, and an ultrasonic treatment apparatus can be independently used as has been described in the first embodiment.

### Second Modification of Second Embodiment

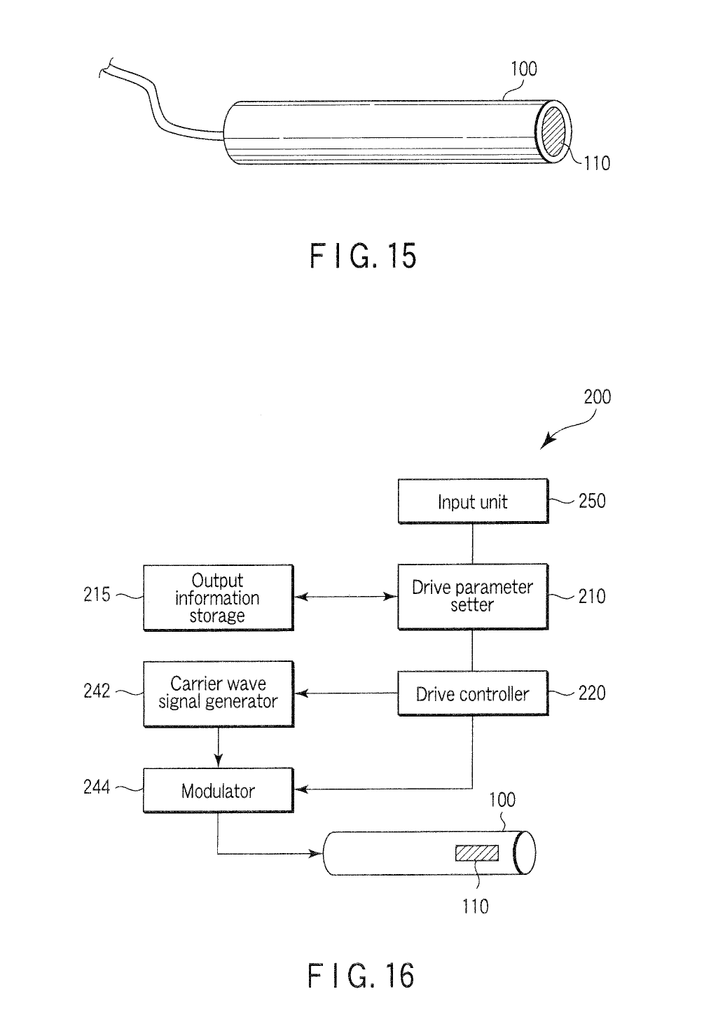

A second modification of the second embodiment is described with reference to the drawings. Here, the difference between the present modification and the second embodiment is described, and the same parts as those in the second embodiment are provided with the same reference numbers and are not described. The tip portion 100 according to the second embodiment is cylindrical, and the ultrasonic wave emitter 110 is disposed in a part of the circumferential surface of the tip portion 100. In contrast, in a tip portion 100 according to this modification, an ultrasonic wave emitter 110 is disposed in the distal face of the tip portion 100, as shown in FIG. 15.

As a result of the modified shape of the tip portion 100, an endoscopic ultrasonic diagnostic treatment apparatus 400 according to the present modification does not have the rotation controller 443 and the unshown rotation device. Instead, the endoscopic ultrasonic diagnostic treatment apparatus 400 may be provided with a known mechanism for changing the radiation angle of ultrasonic waves with respect to the central axis direction of the tip portion 100. For example, the endoscopic ultrasonic diagnostic treatment apparatus 400 can have a mechanism for physically changing the direction of the ultrasonic wave radiation surface of a cMUT array 320. Moreover, the endoscopic ultrasonic diagnostic treatment apparatus 400 can be configured to change the radiation direction of ultrasonic waves by varying the angle of a head 318 of the cMUT 310 as in the first modification of the second embodiment. The endoscopic ultrasonic diagnostic treatment apparatus 400 can also be configured so that the direction of ultrasonic waves emitted from the cMUT array 320 may be changed by a phased array. Each case is essentially not different from the second embodiment. According to the endoscopic ultrasonic diagnostic treatment apparatus 400 having such a configuration, advantages similar to the advantages of the endoscopic ultrasonic diagnostic treatment apparatus 400 according to the second embodiment can also be obtained.

The endoscopic ultrasonic diagnostic treatment apparatus is described by way of example in the second embodiment. However, if the ultrasonic probe 420 and the endoscopic device 460 are modified, a laparoscope, an intraoperative apparatus, an extracorporeal apparatus and other forms of apparatuses can be produced using a similar configuration.

### Third Embodiment

A third embodiment of the present invention is described with reference to the drawings. Here, the difference between the third embodiment and the first embodiment is described, and the same parts as those in the first embodiment are provided with the same reference numbers and are not described. In the ultrasonic treatment apparatus according to the first embodiment, the signals generated by the first signal generator 232 and the second signal generator 234 are added together by the adder 236, and the signal represented by Equation (1) is thereby generated. In contrast, in the present embodiment, a signal similar to that in the first embodiment is generated by amplitude modulation.

In an ultrasonic treatment apparatus according to the present embodiment, an ultrasonic treatment apparatus controller 200 has a drive parameter setter 210, an output information storage 215, a drive controller 220, a carrier wave signal generator 242 and a modulator 244, as shown in FIG. 16. Here, as in the first embodiment, the drive parameter setter 210 reads frequency information from the output information storage 215 on the basis of an input from an input unit 250, calculates various parameters of ultrasonic waves emitted from an ultrasonic wave emitter 110, and outputs the parameters to the drive controller 220.

On the basis of the parameters input from the drive parameter setter 210, the drive controller 220 outputs an instruction for the carrier wave signal generator 242 to generate a signal, and outputs an instruction for the modulator 244 to modulate the signal. In accordance with the instruction input from the drive controller 220, the carrier wave signal generator 242 generates a carrier wave signal, and outputs the carrier wave signal to the modulator 244. In accordance with the instruction input from the drive controller 220, the modulator 244 modulates the carrier wave signal input from the carrier wave signal generator 242. The modulator 244 outputs, to the ultrasonic wave emitter 110, a drive signal which is the signal generated by the modulation. The modulator 244 is a general modulation circuit. For example, the carrier wave signal generator 242 and the modulator 244 function as drive signal generators for generating the drive signal by amplitude modulation.

The operation of the ultrasonic treatment apparatus according to the present embodiment is described. For example, a frequency fₘ of modulated waves is stored in the output information storage 215 in association with an object to be irradiated with ultrasonic waves. A frequency f_{c} of carrier waves is stored in the output information storage 215 in association with the distance (depth) between the ultrasonic wave emitter 110 and the object to be irradiated with ultrasonic waves. In addition, information regarding the ultrasonic waves emitted by the ultrasonic wave emitter 110, such as the strength of the ultrasonic waves is stored in the output information storage 215.

In accordance with an input from the input unit 250, the drive parameter setter 210 reads the information stored in the output information storage 215. On the basis of the read information, the drive parameter setter 210 determines a signal x(t) output by the modulator 244, i.e., a signal for emitting ultrasonic waves from the ultrasonic wave emitter 110, in accordance with the object to be irradiated with ultrasonic waves and the distance to the object. The signal x(t) output by the modulator 244 is represented by, for example, Equation (2).

x(t)=A cos(ω_{c}t)cos(ωₘt) (2)

where ωₘ=2PIfₘ, ω_{c}=2PIf_{c}. As in Equation (1), in this example, x(t)=A cos(ω_{c}t) corresponds to carrier waves, and x(t)=cos(ωₘt) corresponds to modulated waves. The drive parameter setter 210 outputs, to the drive controller 220, the determined signal for emitting ultrasonic waves from the ultrasonic wave emitter 110, i.e., information on the signal x(t) output by the modulator 244. For example, in order to burst microbubbles, fₘ is about 1 to 2 MHz.

On the basis of the signal input from the drive parameter setter 210, the drive controller 220 outputs an instruction for the carrier wave signal generator 242 to generate a signal, and outputs an instruction for the modulator 244 to modulate the signal.

In accordance with the input from the drive controller 220, the carrier wave signal generator 242 generates a signal. For example, in Equation (2), the carrier wave signal generator 242 generates a signal x(t)=A cos(ω_{c}t) as carrier waves. The carrier wave signal generator 242 outputs the generated signal to the modulator 244.

The carrier waves from the carrier wave signal generator 242 are input to the modulator 244. On the basis of the input from the drive controller 220, the modulator 244 generates a modulated wave signal, and modulates the carrier waves by this modulating signal. For example, in Equation (2), the modulator 244 generates a signal x(t)=cos(ωₘt) as modulated waves, and modulates the carrier waves by this modulating signal. The modulator 244 outputs a resultant drive signal to the ultrasonic wave emitter 110.

As in the first embodiment, when this ultrasonic treatment apparatus is used, the ultrasonic wave emitter 110 of the tip portion 100 is brought into contact with a subject (sound propagating medium) to be irradiated with ultrasonic waves. There may be an acoustic matching layer for matching acoustic impedance between the ultrasonic wave emitter 110 and the subject, or the ultrasonic wave emitter 110 and the subject may be in direct contact. Each of cMUTs 310 constituting a cMUT array 320 of the ultrasonic wave emitter 110 vibrates a head 318 in response to the drive signal input from the modulator 244. As a result, ultrasonic waves represented by, for example, Equation (2) and corresponding to the signal x(t) output by the modulator 244 are emitted from the ultrasonic wave emitter 110. The radiation of the ultrasonic waves is equal to the fact that ultrasonic waves having the frequency of the modulated waves are propagated to a target position by the carrier waves. That is, the ultrasonic waves having the frequency of the modulated waves are self-demodulated at the target position by radiation of the ultrasonic waves. As a result, the ultrasonic waves having the frequency of the modulated waves are applied to the target position.

According to the ultrasonic treatment apparatus of the present embodiment, ultrasonic waves having a frequency suited for an object can be radiated regardless of the configuration of the ultrasonic wave emitter 110, as in the first embodiment. A frequency of the carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

### Modification of Third Embodiment

As in the first modification of the second embodiment described with reference to FIG. 12B, the ultrasonic treatment apparatus according to the third embodiment may be configured so that heads 318 of cMUTs 310 have different slopes. As in the first modification of the second embodiment described with reference to FIG. 14, the ultrasonic treatment apparatus may be configured so that the emission direction of ultrasonic waves can be changed by a phased array. Moreover, the endoscopic ultrasonic diagnostic treatment apparatus 400 described in the second embodiment may be configured by use of the ultrasonic treatment apparatus according to the present embodiment or the above-mentioned modifications. Such a modification can also provide advantages similar to the advantages described above.

### Fourth Embodiment

A forth embodiment of the present invention is described with reference to the drawings. Here, the difference between the forth embodiment and the first embodiment is described, and the same parts as those in the first embodiment are provided with the same reference numbers and are not described. In the ultrasonic treatment apparatus according to the first embodiment, the adder 236 adds together the signal of the frequency f₁ generated by the first signal generator 232 and the signal of the frequency f₂ generated by the second signal generator 234, thereby generating the signal represented by Equation (1).

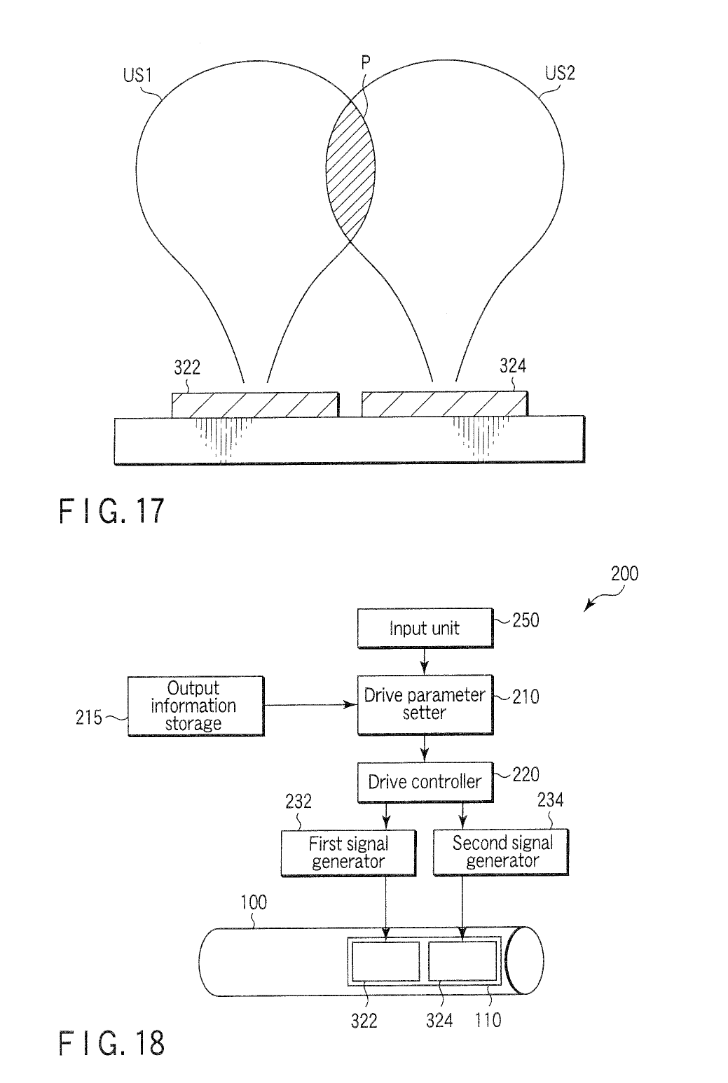

In contrast, in the present embodiment, cMUTs 310 which are ultrasonic wave generating elements of a cMUT array 320 of an ultrasonic wave emitter 110 are separated into two groups. As shown in FIG. 17, ultrasonic waves US1 of the frequency f₁ are generated from a first cMUT array 322 which is a first group, and ultrasonic waves US2 of the frequency f₂ are generated from a second cMUT array 324 which is a second group. As a result, in a space where both kinds of the ultrasonic waves are radiated, a harmonic or combination tone is generated by the nonlinearity of a medium that conveys acoustic waves, in a superposed space P (a shaded region in FIG. 17) where the ultrasonic waves US1 of the frequency f₁ and the ultrasonic waves US2 of the frequency f₂ are superposed on each other. Thus, in the superposed space P, there is produced a condition substantially similar to the condition in which the ultrasonic waves generated by the signal x(t) represented by Equation (1) in the first embodiment are radiated. That is, ultrasonic waves having a frequency Δf=|f₁−f₂| are generated in the superposed space P. Such an effect is referred to as a parametric effect.

The configuration of an ultrasonic treatment apparatus according to the present embodiment is shown in FIG. 18. An ultrasonic treatment apparatus controller 200 of this ultrasonic treatment apparatus includes a drive parameter setter 210, an output information storage 215, a drive controller 220, a first signal generator 232 and a second signal generator 234, as in the first embodiment. The function of each component is similar to that in the first embodiment. However, in the present embodiment, the adder 236 that is present in the first embodiment is not provided. In the present embodiment, the first signal generator 232 outputs the generated signal of the frequency f₁ to the first cMUT array 322 of the ultrasonic wave emitter 110 as a drive signal. The second signal generator 234 outputs the generated signal of the frequency f₂ to the second cMUT array 324 of the ultrasonic wave emitter 110 as a drive signal.

In response to the drive signal input from the first signal generator 232, each of the cMUTs 310 constituting the first cMUT array 322 vibrates the head 318 to radiate ultrasonic waves of the frequency f₁. In response to the drive signal input from the second signal generator 234, each of the cMUTs 310 constituting the second signal generator 234 vibrates the head 318 to radiate ultrasonic waves of the frequency f₂. Among the cMUTs 310 constituting the cMUT array 320, any of the cMUTs 310 can function as the first cMUT array 322, and any of the cMUTs 310 can function as the second cMUT array 324.

For example, the first cMUT array 322 functions as at least one ultrasonic wave emitter driven by a first drive signal. For example, the second cMUT array 324 functions as at least another ultrasonic wave emitter driven by a second drive signal.

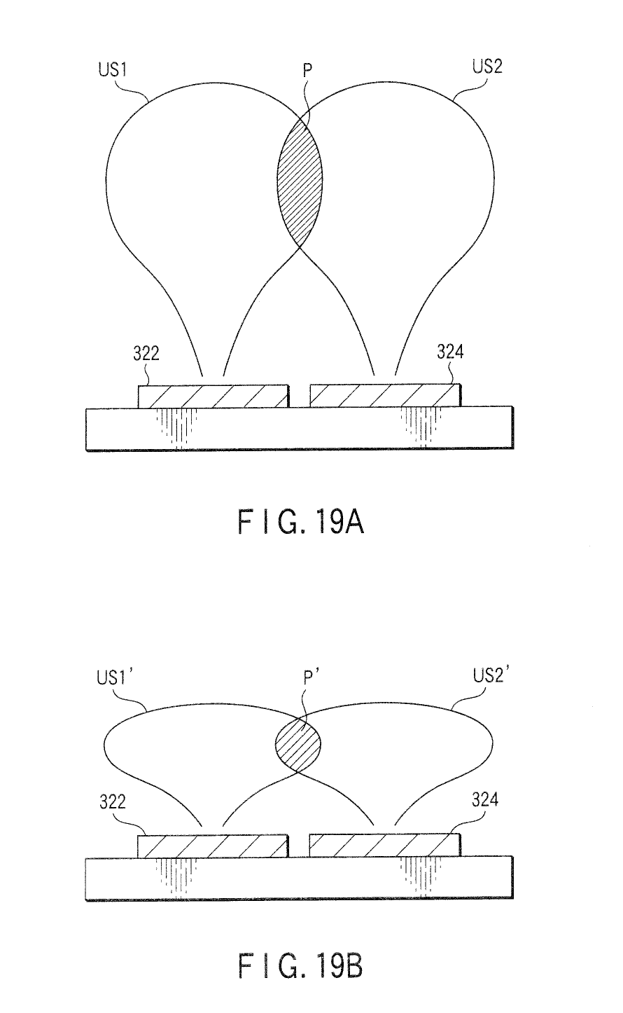

When such a configuration is used in operation, the ultrasonic waves are propagated as in the schematic diagrams of FIGS. 19A and 19B. For example, the frequency f₁ of the ultrasonic waves US1 emitted by the first cMUT array 322 is 6 MHz, and the frequency f₂ of the ultrasonic waves US2 emitted by the second cMUT array 324 is 5 MHz. In consequence, the propagation region of the ultrasonic waves is as shown in FIG. 19A. Here, a frequency Δf of the ultrasonic waves generated in the superposed space P is 1 MHz. On the other hand, for example, the frequency f₁ of ultrasonic waves US1′ emitted by the first cMUT array 322 is 21 MHz, and the frequency f₂ of the ultrasonic waves US2′ emitted by the second cMUT array 324 is 20 MHz. In consequence, the propagation region of the ultrasonic waves is as shown in FIG. 19B because a higher frequency leads to more attenuating so that the range of the ultrasonic waves is smaller. In this case as well, the frequency Δf of the ultrasonic waves generated in a superposed space P′ is 1 MHz. Thus, in each case, the ultrasonic waves of Δf=1 MHz are generated in the superposed space. On the other hand, the region where the ultrasonic waves of Δf=1 MHz are generated varies depending on the combination of the frequencies f₁ and f₂.

According to the ultrasonic treatment apparatus of the present embodiment, ultrasonic waves having a frequency suited for an object can be radiated regardless of the configuration of the ultrasonic wave emitter 110, as in the first embodiment. A frequency of the carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

### First Modification of Fourth Embodiment

A first modification of the forth embodiment is described with reference to the drawings. Here, the difference between the present modification and the forth embodiment is described, and the same parts as those in the forth embodiment are provided with the same reference numbers and are not described.

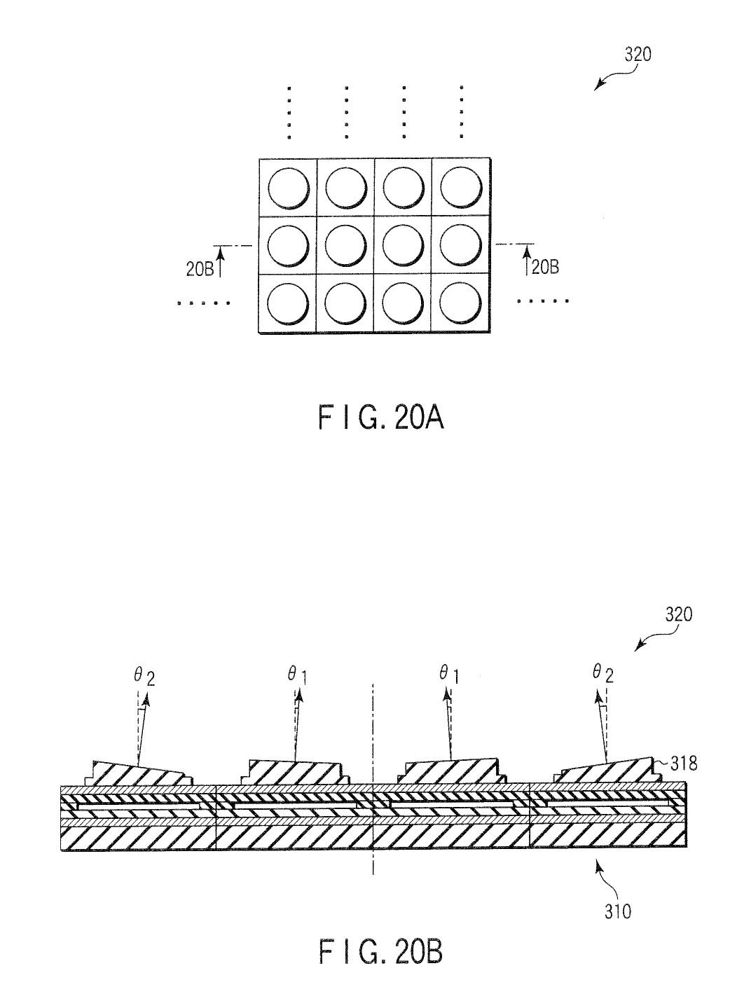

In the fourth embodiment, the ultrasonic wave emitting surfaces of the ultrasonic wave emitters 110 described with reference to FIG. 17 are parallel to each other. In contrast, the ultrasonic wave emitting surfaces of ultrasonic wave emitters 110 according to this modification are sloped, as shown in a plane view of FIG. 20A and a sectional view of FIG. 20B through 20B-20B of FIG. 20A. For example, as shown in FIG. 20B, heads 318 of cMUTs 310 are designed to be differently sloped symmetrically with respect to a central line indicated by a dashed-dotted line. The slopes are angled to focus emitted ultrasonic waves.

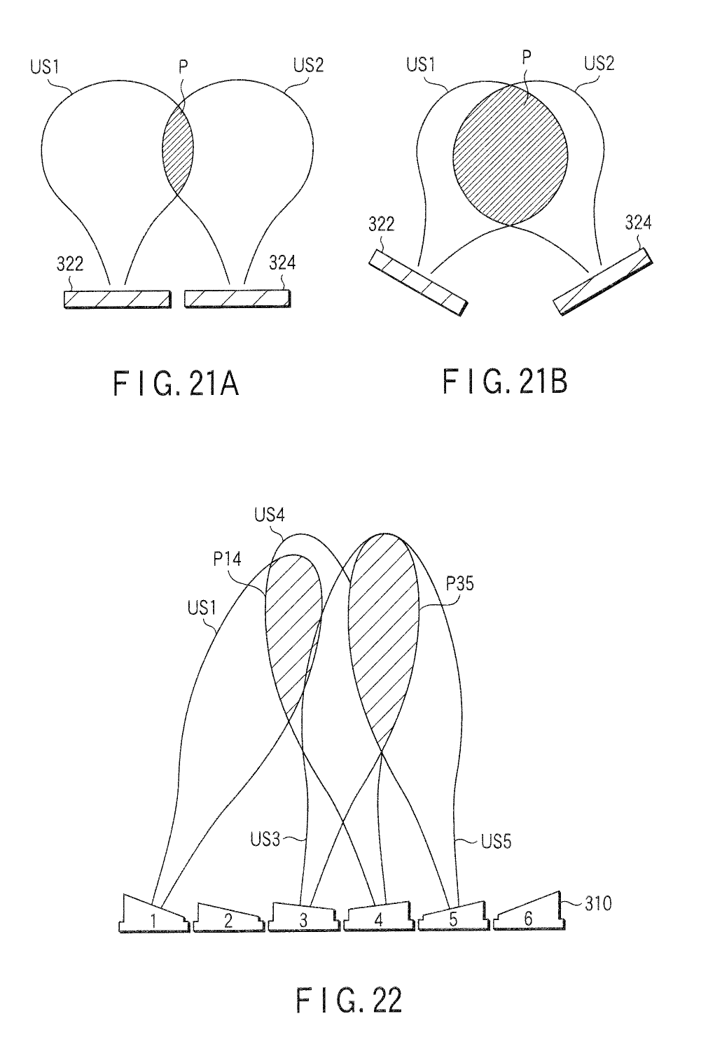

Ultrasonic waves US1 of a frequency f₁ emitted from first group ultrasonic wave generating elements and ultrasonic waves US2 of a frequency f₂ emitted from second group ultrasonic wave generating elements are superposed on each other in a superposed space P. The superposed space P is larger as shown in FIG. 21B when emitting surfaces are designed to be sloped as has been described with reference to FIGS. 20A and 20B than when emitting surfaces are parallel to one other as shown in FIG. 21A. Consequently, this ultrasonic treatment apparatus can efficiently use the energy of emitted ultrasonic waves.

Among cMUTs 310, any of the cMUTs 310 can serve as a first cMUT array 322, and any of the cMUTs 310 can serve as a second cMUT array 324. The operation is the same as that of the ultrasonic treatment apparatus according to the fourth embodiment. According to this modification, advantages similar to the advantages according to the fourth embodiment can also be obtained.

### Second Modification of Fourth Embodiment

As in the first modification of the second embodiment described with reference to FIG. 12B, the ultrasonic treatment apparatus according to the fourth embodiment may be configured so that the heads 318 of the cMUTs 310 are differently sloped to enable ultrasonic waves to be radiated in various directions. When the heads 318 of the cMUTs 310 are configured to be differently sloped, a superposed space P where the ultrasonic waves US1 and the ultrasonic waves US2 are superposed on each other can be formed at various positions by properly selecting the first group ultrasonic wave generating elements for emitting the ultrasonic waves US1 of the frequency f₁ and the second group ultrasonic wave generating elements for emitting the ultrasonic waves US2 of the frequency f₂.

For example, as schematically shown in FIG. 22, ultrasonic waves US1 are emitted from the cMUT 310 indicated by “1”, and ultrasonic waves US4 are emitted from the cMUT 310 indicated by “4”, such that ultrasonic waves having a difference tone are generated in a superposed space P14. On the other hand, ultrasonic waves US3 are emitted from the cMUT 310 indicated by “3”, and ultrasonic waves US5 are emitted from the cMUT 310 indicated by “5”, such that ultrasonic waves having a difference tone are generated in a superposed space P35. Thus, the target position can be changed by selecting the first group ultrasonic wave generating elements and the second group ultrasonic wave generating elements.

As in the first modification of the second embodiment described with reference to FIG. 14, the ultrasonic treatment apparatus may be configured so that the emission direction of ultrasonic waves can be changed by a phased array. Moreover, the endoscopic ultrasonic diagnostic treatment apparatus 400 described in the second embodiment may be configured by use of the ultrasonic treatment apparatus according to the present embodiment or the above-mentioned modifications. Such a modification can also provide advantages similar to the advantages described above.

### Fifth Embodiment

A fifth embodiment of the present invention is described with reference to the drawings. Here, the difference between the fifth embodiment and the first embodiment is described, and the same parts as those in the first embodiment are provided with the same reference numbers and are not described.

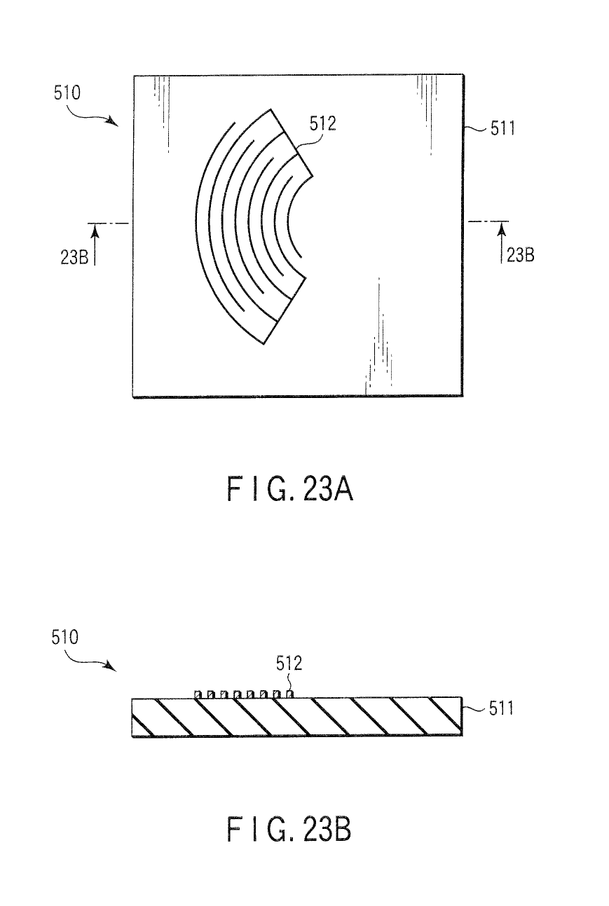

In the ultrasonic treatment apparatus according to the first embodiment, the cMUT array 320 is used as the ultrasonic wave emitter 110. In contrast, a focal interdigital transducer (F-IDT) 510 is used as an ultrasonic wave emitter 110 of the ultrasonic treatment apparatus according to the present embodiment. In the interdigital transducer 510, an arc-shaped interdigital electrode 512 is formed on a Y-cut Z-propagation lithium niobate (LiNbO₃) substrate 511, as shown in a plane view of FIG. 23A and a sectional view of FIG. 23B. The operational principle of the IDT is described in “Maezawa and Kamakura (2009). Acoustic streaming generated by switching radiation modes for SAW device and its application to liquid mixing. Inst. Electron, Inform. Commun. Eng Tech. Rep., Vol. 109(17), 17-22,” the entire contents of which are incorporated herein by reference. The configuration of the ultrasonic treatment apparatus according to the present embodiment is similar to the configuration according to the first embodiment described with reference to FIG. 1 except that the F-IDT is used as the ultrasonic wave emitter 110.

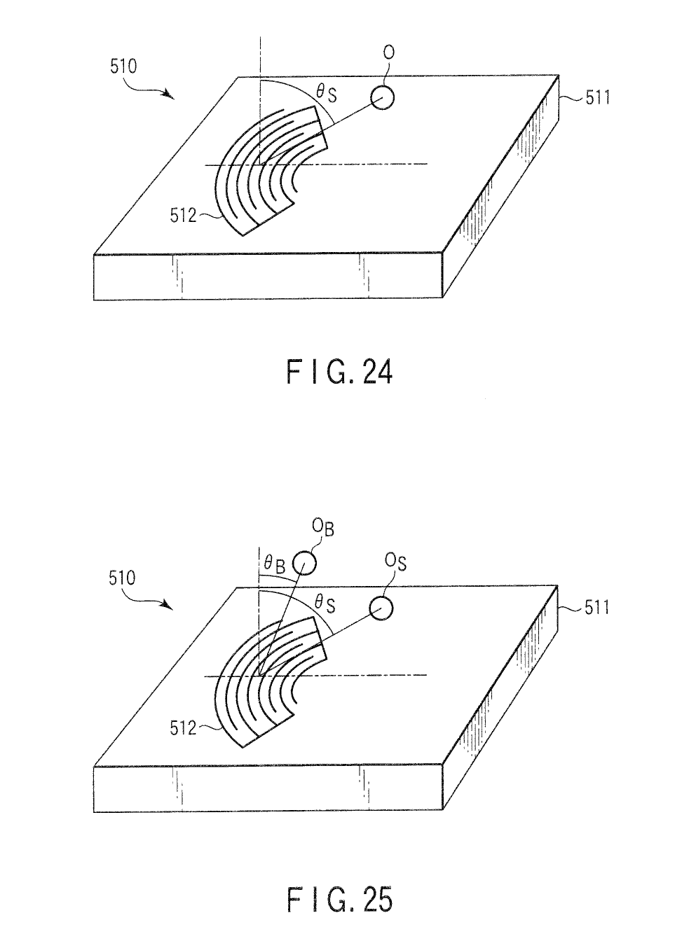

The ultrasonic wave emitter 110 comprising the F-IDT 510 is in contact with a subject (sound propagating medium) in operation. When a voltage is applied to the interdigital electrode 512 of the F-IDT 510, the interdigital electrode vibrates, and surface acoustic waves (SAWs) are generated on the surface of the lithium niobate substrate 511. The SAWs propagate along the surface of the lithium niobate substrate 511, and are then radiated to the subject. The frequency of the SAWs is about several ten MHz. As the interdigital electrode 512 is arc-shaped, the propagated ultrasonic waves are focused on a focus O as shown in FIG. 24. That is, the energy of the ultrasonic waves can be concentrated on the focus O. Here, the focus O is at a position corresponding to the center of a sector form of the interdigital electrode 512. An ultrasonic wave radiation angle θs with the normal to the surface of the lithium niobate substrate 511 where the interdigital electrode 512 is formed is determined by a ratio between the propagation velocity of the ultrasonic waves in the lithium niobate substrate 511 and the propagation velocity of the ultrasonic waves in the subject. There are a relatively small number of pairs, for example, several pairs to several ten pairs of engaged electrodes, so that an output frequency band is broader.

The F-IDT 510 which emits ultrasonic waves having a high frequency of about several ten MHz is used to apply, to a desired position (depth), ultrasonic waves of, for example, 1 to 2 MHz which is the resonance frequency of the microbubbles. Thus, in the present embodiment, the self-demodulation effect is used as in the first embodiment. Accordingly, an ultrasonic treatment apparatus controller 200 for controlling and driving the F-IDT 510 is the same as the ultrasonic treatment apparatus controller 200 according to the first embodiment.

The ultrasonic treatment apparatus controller 200 includes a drive parameter setter 210, an output information storage 215, a drive controller 220, a first signal generator 232, a second signal generator 234 and an adder 236. In response to the operator instruction input from an input unit 250, the drive parameter setter 210 of the ultrasonic treatment apparatus controller 200 determines frequencies of carrier waves and modulated waves in accordance with a target position and an object to be irradiated with ultrasonic waves. For the determined frequencies of the carrier waves and the modulated waves, the drive parameter setter 210 determines the frequency f₁, amplitude A₁ and initial phase θ₁ of a signal generated by the first signal generator 232 and the frequency f₂, amplitude A₂ and initial phase θ₂ of the signal generated by the second signal generator 234. Here, the drive parameter setter 210 uses frequency information stored in the output information storage 215, for example, shown in FIG. 8. The drive parameter setter 210 outputs the determined f₁, f₂, A₁, A₂, θ₁ and θ₂ to the drive controller 220.

On the basis of the value input from the drive parameter setter 210, the drive controller 220 outputs an instruction for the first signal generator 232 to generate a signal having the frequency f₁, amplitude A₁ and initial phase θ₁. The drive controller 220 also outputs an instruction for the second signal generator 234 to generate a signal having the frequency f₂, amplitude A₂ and initial phase θ₂. On the basis of the instructions input from the drive controller 220 to generate ultrasonic waves, the first signal generator 232 and the second signal generator 234 respectively generate signals and output the signals to the adder 236. The adder 236 adds together the signals input from the first signal generator 232 and the second signal generator 234, and outputs a drive signal represented by Equation (1) to the ultrasonic wave emitter 110.

Consequently, this ultrasonic treatment apparatus can apply ultrasonic waves having a desired frequency to the target position as shown in FIG. 24 by the principle and operation similar to those described in the first embodiment. That is, when the drive signal is applied to the interdigital electrode 512 of the F-IDT 510, the interdigital electrode vibrates, and SAWs are generated on the surface of the lithium niobate substrate 511. The SAWs are radiated to the subject. The radiated ultrasonic waves focus on the focus O. If the focus O is set at the target position, self-demodulated ultrasonic waves are generated at the target position.

Furthermore, when driven at a frequency which is about 1.5 to 2 times the center frequency of the SAWs, the F-IDT 510 generates strong bulk acoustic waves (BAWs). While the SAWs propagate along the surface of the substrate and are applied to the subject, the BAWs propagate through the lithium niobate substrate 511 from the surface in which the interdigital electrode 512 is formed. The BAWs which have propagated through the lithium niobate substrate 511 are reflected on the rear side of the surface in which the interdigital electrode 512 is formed, and return to the surface in which the interdigital electrode 512 is formed. From this surface, ultrasonic waves are then radiated to the subject. In this case, an angle θ_{B} formed by the radiation direction of the ultrasonic waves with the normal to the lithium niobate substrate 511 is smaller than an angle θ_{S} formed by the radiation direction of the ultrasonic waves derived from the SAWs with the normal, as shown in FIG. 25.

In the ultrasonic treatment apparatus according to the present embodiment, the drive parameter setter 210 properly selects a frequency input to the interdigital electrode 512, so that the SAWs or BAWs can be generated. The BAWs can also be excited by raising or dropping a drive frequency with respect to the center frequency of the SAWs. In such a manner, the angle of the ultrasonic wave radiation to the subject, that is, the focal position can be changed as shown in FIG. 25. Thus, this ultrasonic treatment apparatus is capable of changing the target position to be irradiated with ultrasonic waves.

Y-cut Z-propagation lithium niobate (YZ—LiNbO₃) is not exclusively used for the lithium niobate substrate 511. For example, 128°-rotated Y-cut Z-propagation lithium niobate (128YX—LiNbO₃) may be used. It is known that both of the Y-cut Z-propagation and 128°-rotated Y-cut X-propagation provide sections that efficiently generate ultrasonic waves. If the SAWs and/or the BAWs can be generated, PZT or ZnO may be used instead of lithium niobate.

According to the ultrasonic treatment apparatus of the present embodiment, ultrasonic waves of, for example, 1 to 2 MHz which is the resonance frequency of the microbubbles can be radiated even by the F-IDT having a high output frequency of several ten MHz, as in the first embodiment. According to the ultrasonic treatment apparatus of the present embodiment, ultrasonic waves having a frequency suited for an object can be radiated regardless of the configuration of the ultrasonic wave emitter 110. A frequency of the carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

### First Modification of Fifth Embodiment

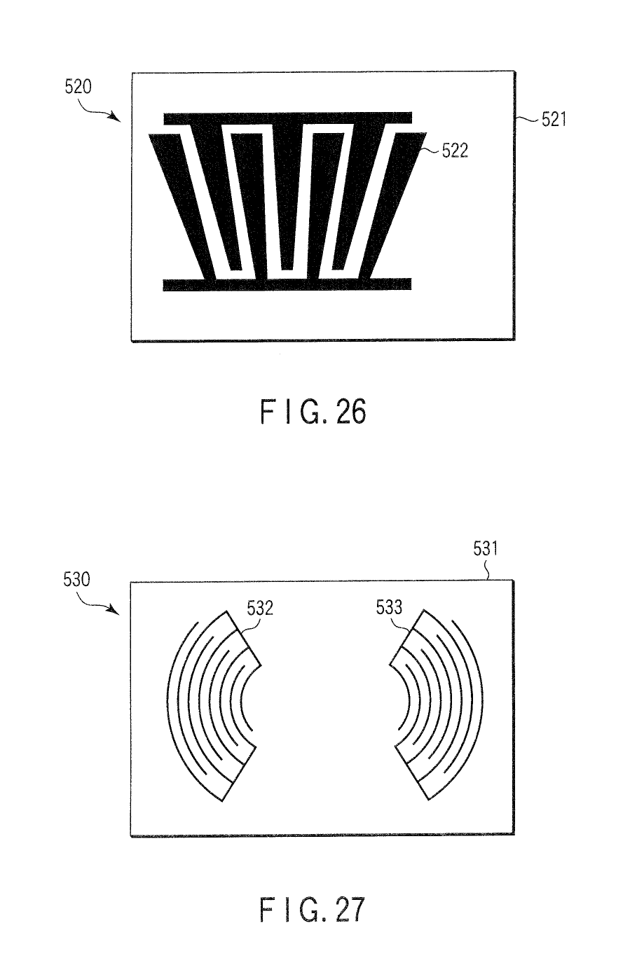

A first modification of the fifth embodiment is described. The ultrasonic wave emitter 110 according to the fifth embodiment comprises the F-IDT 510 in which the arc-shaped interdigital electrode 512 is formed on the lithium niobate substrate 511. In contrast, an ultrasonic wave emitter 110 according to the present embodiment comprises an IDT 520. As shown in FIG. 26, the IDT 520 has a configuration in which an interdigital electrode 522 that varies in width depending on the position is formed on a lithium niobate substrate 521. The width of the interdigital electrode 522 varies depending on the position, so that the SAWs and BAWs that are generated depending on the position of the IDT 520 are different in frequency. That is, the frequency of generated acoustic waves is higher in thinner parts of the interdigital electrode 522 (lower parts in FIG. 26), and the frequency of generated acoustic waves is lower in thicker parts of the interdigital electrode 522 (upper parts in FIG. 26).

Such a configuration allows the output frequency band of the IDT 520 that configures the ultrasonic wave emitter 110 to be broader. Here, if there are a relatively small number of pairs, for example, about 2 to 10 pairs of engaged electrodes, the output frequency band of the IDT 520 is further broadened.

### Second Modification of Fifth Embodiment

The endoscopic ultrasonic diagnostic treatment apparatus 400 described in the second embodiment can be configured by use of the ultrasonic treatment apparatus according to the fifth embodiment or its first modification. The endoscopic ultrasonic diagnostic treatment apparatus 400 configured by use of the ultrasonic treatment apparatus according to the fifth embodiment or its first modification can provide operation and advantages similar to those in the second embodiment.

### Sixth Embodiment

A sixth embodiment of the present invention is described. In an ultrasonic treatment apparatus according to the present embodiment, the F-IDT 510 described with reference to FIGS. 23A and 238 or the IDT 520 described with reference to FIG. 26 is used as an ultrasonic wave emitter 110, similarly to the ultrasonic wave emitter 110 according to the fifth embodiment. An ultrasonic treatment apparatus controller 200 in the ultrasonic treatment apparatus according to the present embodiment has the configuration described with reference to FIG. 16 similarly to the ultrasonic treatment apparatus controller 200 according to the third embodiment, and controls and drives the ultrasonic wave emitter 110 by a method that uses amplitude modulation.

For example, a frequency fₘ of modulated waves is stored in an output information storage 215 in association with an object to be irradiated with ultrasonic waves. A frequency f_{c} of carrier waves is stored in the output information storage 215 in association with the distance (depth) between an ultrasonic wave emitter 110 and the object to be irradiated with ultrasonic waves. In addition, information regarding the ultrasonic waves emitted by the ultrasonic wave emitter 110, such as the strength of the ultrasonic waves is stored in the output information storage 215. In accordance with an input from an input unit 250, the drive parameter setter 210 reads the information stored in the output information storage 215. On the basis of the read information, the drive parameter setter 210 determines a signal of ultrasonic waves to be emitted from the ultrasonic wave emitter 110, in accordance with the object to be irradiated with ultrasonic waves and the distance to the object. The drive parameter setter 210 outputs the determined ultrasonic wave signal to a drive controller 220.

On the basis of the signal information input from the drive parameter setter 210, the drive controller 220 outputs an instruction for a carrier wave signal generator 242 to generate a signal, and outputs an instruction for a modulator 244 to modulate the signal. In accordance with the input from the drive controller 220, the carrier wave signal generator 242 generates a carrier wave signal. The carrier wave signal generator 242 outputs the generated signal to the modulator 244. The carrier wave signal is input to the modulator 244 from the carrier wave signal generator 242. On the basis of the input from the drive controller 220, the modulator 244 generates a modulated wave signal. The modulator 244 modulates, by the generated modulating signal, the carrier waves input from the carrier wave signal generator 242. The modulator 244 outputs a resultant drive signal to the ultrasonic wave emitter 110. In accordance with the drive signal input from the modulator 244, the F-IDT 510 or the IDT 520 that configures the ultrasonic wave emitter 110 generates ultrasonic waves.

In this manner, the ultrasonic wave emitter 110 emits ultrasonic waves in accordance with, for example, the signal x(t) which is output by the modulator 244 and which is represented by, for example, Equation (2). As a result, the ultrasonic waves propagate to a target position on the carrier waves, and the ultrasonic waves having the frequency of the modulated waves are self-demodulated at the target position. That is, the ultrasonic waves having the frequency of the modulated waves are applied to the target position.

According to the ultrasonic treatment apparatus of the present embodiment having the configuration described above, ultrasonic waves having a frequency suited for an object can be radiated regardless of the configuration of the ultrasonic wave emitter 110, as in the third embodiment and the fifth embodiment. A frequency of the carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

The endoscopic ultrasonic diagnostic treatment apparatus 400 described in the second embodiment can be configured by use of the ultrasonic treatment apparatus according to the sixth embodiment. The endoscopic ultrasonic diagnostic treatment apparatus 400 configured by use of the ultrasonic treatment apparatus according to the sixth embodiment can provide operation and advantages similar to those in the second embodiment.

### Seventh Embodiment

A seventh embodiment of the present invention is described with reference to the drawings. In an ultrasonic treatment apparatus according to the present embodiment, an IDT is used as an ultrasonic wave emitter 110, similarly to the ultrasonic wave emitter 110 according to the fifth embodiment. An ultrasonic treatment apparatus controller 200 in the ultrasonic treatment apparatus according to the present embodiment has the configuration described with reference to FIG. 18 similarly to the ultrasonic treatment apparatus controller 200 according to the fourth embodiment. The ultrasonic treatment apparatus controller 200 controls and drives the ultrasonic wave emitter 110 by a method that uses a parametric effect attributed to a difference tone.

An F-IDT 530 shown in FIG. 27 is used as the ultrasonic wave emitter 110 according to the present embodiment. A lithium niobate substrate 531 similar to the lithium niobate substrate 511 of the F-IDT 510 described in the fifth embodiment is used in the F-IDT 530. Two arc-shaped interdigital electrodes similar to the interdigital electrode 512 of the F-IDT 510 are formed on the lithium niobate substrate 531. That is, a first interdigital electrode 532 and a second interdigital electrode 533 are formed on the lithium niobate substrate 531 so that the central directions of sector form of these electrodes face each other. A first signal generator 232 of the ultrasonic treatment apparatus controller 200 is connected to the first interdigital electrode 532. A second signal generator 234 is connected to the second interdigital electrode 533. For example, the first interdigital electrode 532 functions as at least one interdigital electrode to which a first drive signal is input. For example, the second interdigital electrode 533 functions as at least another interdigital electrode to which a second drive signal is input.

The operation of the ultrasonic treatment apparatus according to the present embodiment having such a configuration is described. The ultrasonic treatment apparatus controller 200 operates as in the fourth embodiment. In response to an operator instruction input from an input unit 250, a drive parameter setter 210 of the ultrasonic treatment apparatus controller 200 determines frequencies of carrier waves and modulated waves in accordance with a target position and an object to be irradiated with ultrasonic waves. For the determined frequencies of the carrier waves and the modulated waves, the drive parameter setter 210 determines the frequency f₁, amplitude A₁ and initial phase θ₁ of a signal generated by the first signal generator 232 and the frequency f₂, amplitude A₂ and initial phase θ₂ of the signal generated by the second signal generator 234. Here, the drive parameter setter 210 uses frequency information stored in an output information storage 215. The drive parameter setter 210 outputs the determined f₁, f₂, A₁, A₂, θ₁ and θ₂ to the drive controller 220.

On the basis of the value input from the drive parameter setter 210, the drive controller 220 outputs an instruction for the first signal generator 232 to generate a signal having the frequency f₁, amplitude A₁ and initial phase θ₁. The drive controller 220 also outputs an instruction for the second signal generator 234 to generate a signal having the frequency f₂, amplitude A₂ and initial phase θ₂. The first signal generator 232 outputs the generated signal to the first interdigital electrode 532. The second signal generator 234 likewise outputs the generated signal to the second interdigital electrode 533.

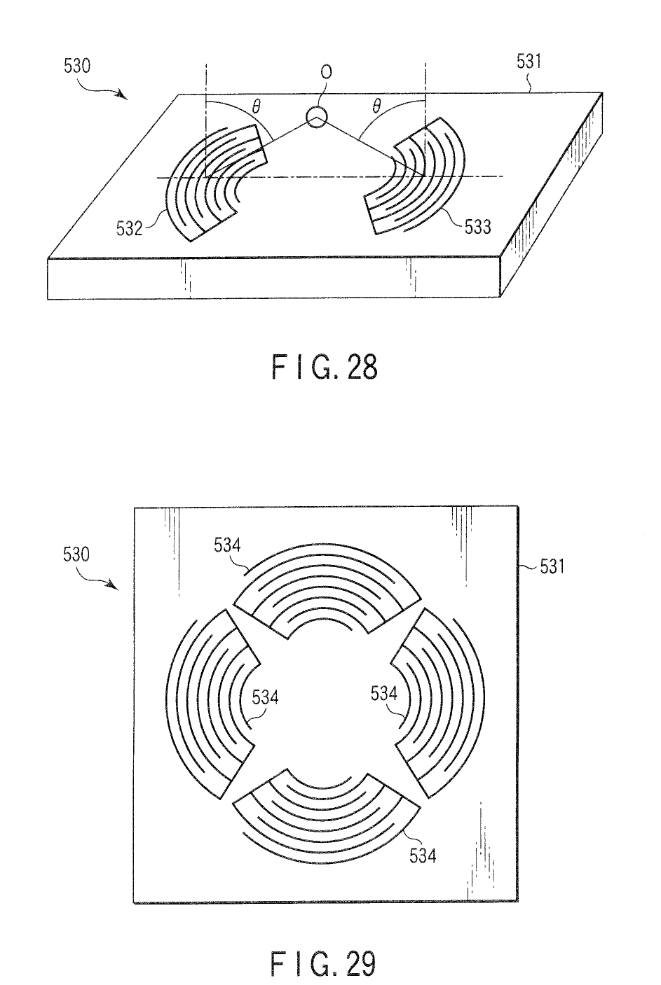

As a result, the first interdigital electrode 532 and the second interdigital electrode 533 of the F-IDT 530 vibrate, and each electrode generates SAWs or BAWs. The generated SAWs or BAWs are emitted to a subject as shown in FIG. 28. Ultrasonic waves emitted from the first interdigital electrode 532 and ultrasonic waves emitted from the second interdigital electrode 533 overlap at a focus O. Consequently, ultrasonic waves having a frequency Δf=|f₁−f₂| which is a difference tone are generated at the focus O by the parametric effect as in the fourth embodiment.

According to the ultrasonic treatment apparatus of the present embodiment, ultrasonic waves having a frequency suited for an object can be radiated regardless of the configuration of the ultrasonic wave emitter 110, as in the fourth embodiment and the fifth embodiment. A frequency of carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

The F-IDT 530 according to the present embodiment can be configured so that three or more interdigital electrodes 534 are formed on the lithium niobate substrate 531 as shown in FIG. 29. Similar advantages can also be obtained by the F-IDT 530 having such a configuration.

The endoscopic ultrasonic diagnostic treatment apparatus 400 described in the second embodiment can be configured by use of the ultrasonic treatment apparatus according to the seventh embodiment. The endoscopic ultrasonic diagnostic treatment apparatus 400 configured by use of the ultrasonic treatment apparatus according to the seventh embodiment can provide operation and advantages similar to those in the second embodiment.

### Eighth Embodiment

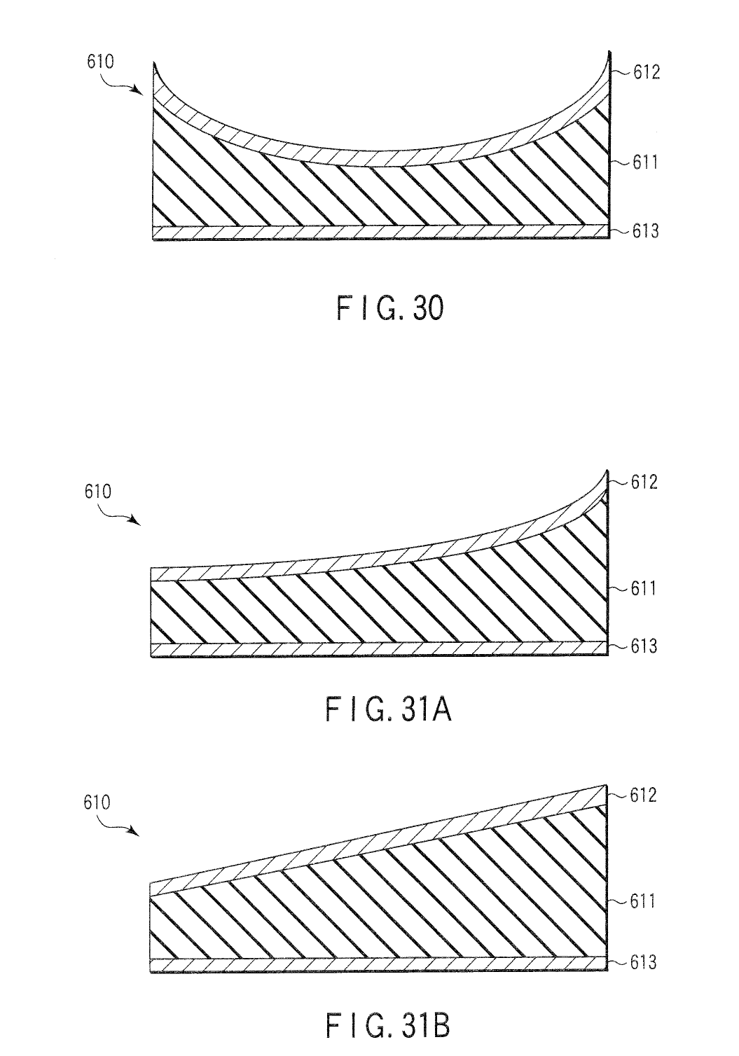

An eighth embodiment of the present invention is described with reference to the drawings. Here, the difference between the eighth embodiment and the first embodiment is described, and the same parts as those in the first embodiment are provided with the same reference numbers and are not described. In the ultrasonic treatment apparatus according to the first embodiment, the cMUT array 320 is used as the ultrasonic wave emitter 110. In contrast, a plano-concave piezoelectric element having a concave upper surface and a planar lower surface shown in FIG. 30 is used as an ultrasonic wave emitter 110 in the ultrasonic treatment apparatus according to the present embodiment.

In an ultrasonic element 610, a ground electrode 612, for example, is formed on the upper surface of a plano-concave piezoelectric element 611, and a signal electrode 613, for example, is formed on the lower surface of the piezoelectric element 611. The piezoelectric element 611 gradually varies in thickness between the central portion and peripheral edge portion, and thus varies in resonance frequency from place to place. Thus, a part that mainly vibrates changes depending on the frequency of an applied voltage. For example, ultrasonic waves having a relatively high frequency are generated in the central portion where the piezoelectric element 611 is thin. Ultrasonic waves having a relatively low frequency are generated in the peripheral edge portion where the piezoelectric element 611 is thick. That is, the output frequency band of the ultrasonic element 610 is broad. The ultrasonic element 610 has, for example, an outside diameter φ of 12 mm, a maximum thickness of 2 mm and a concave surface center thickness of 1.2 mm.

An ultrasonic treatment apparatus controller 200 of the ultrasonic treatment apparatus according to the present embodiment is similar to the ultrasonic treatment apparatus controller 200 according to the first embodiment. The ultrasonic treatment apparatus controller 200 has the configuration described with reference to FIG. 1. Signals generated by a first signal generator 232 and a second signal generator 234 are added together by the adder 236, and the ultrasonic wave emitter 110 is controlled and driven.

More specifically, the ultrasonic treatment apparatus controller 200 has a drive parameter setter 210, an output information storage 215, a drive controller 220, the first signal generator 232, the second signal generator 234 and the adder 236. In response to an operator instruction input from an input unit 250, the drive parameter setter 210 of the ultrasonic treatment apparatus controller 200 determines frequencies of carrier waves and modulated waves in accordance with a target position and an object to be irradiated with ultrasonic waves. For the determined frequencies of the carrier waves and the modulated waves, the drive parameter setter 210 determines the frequency f₁, amplitude A₁ and initial phase θ₁ of a signal generated by the first signal generator 232 and the frequency f₂, amplitude A₂ and initial phase θ₂ of the signal generated by the second signal generator 234. Here, the drive parameter setter 210 uses the frequency information stored in an output information storage 215, for example, shown in FIG. 8. The drive parameter setter 210 outputs the determined f₁, f₂, A₁, A₂, θ₁ and θ₂ to the drive controller 220.

On the basis of the value input from the drive parameter setter 210, the drive controller 220 outputs an instruction for the first signal generator 232 to generate a signal having the frequency f₁, amplitude A₁ and initial phase θ₁. The drive controller 220 also outputs an instruction for the second signal generator 234 to generate a signal having the frequency f₂, amplitude A₂ and initial phase θ₂. On the basis of the instructions input from the drive controller 220 to generate ultrasonic waves, the first signal generator 232 and the second signal generator 234 respectively generate signals and output the generated signals to the adder 236. The adder 236 adds together the inputs from the first signal generator 232 and the second signal generator 234, and outputs a drive signal represented by, for example, Equation (1) to the ultrasonic wave emitter 110. Consequently, this ultrasonic treatment apparatus applies ultrasonic waves having a desired frequency to the target position by the principle and operation similar to those described in the first embodiment, i.e., by using the self-demodulation effect.

According to the ultrasonic treatment apparatus of the present embodiment having the configuration described above, as in the first embodiment, ultrasonic waves of, for example, 1 to 2 MHz which is the resonance frequency of the microbubbles can be radiated even by the ultrasonic wave emitter 110 in which the frequency of output ultrasonic waves has to be high due to the size reduction of the piezoelectric element. Thus, ultrasonic waves having a frequency suited for an object can be radiated regardless of the configuration of the ultrasonic wave emitter 110. A frequency of carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

The piezoelectric element 611 of the ultrasonic element 610 has only to vary in thickness from place to place. The piezoelectric element 611 may be shaped, for example, as shown in FIGS. 31A and 31B. Similar advantages can be obtained in this case as well.

The endoscopic ultrasonic diagnostic treatment apparatus 400 described in the second embodiment can be configured by use of the ultrasonic treatment apparatus according to the eighth embodiment. The endoscopic ultrasonic diagnostic treatment apparatus 400 configured by use of the ultrasonic treatment apparatus according to the eighth embodiment can provide operation and advantages similar to those in the second embodiment.

### Ninth Embodiment

A ninth embodiment of the present invention is described. In an ultrasonic treatment apparatus according to the present embodiment, the ultrasonic element 610 described with reference to FIG. 30 or FIG. 31 is used as an ultrasonic wave emitter 110, similarly to the ultrasonic wave emitter 110 according to the eighth embodiment. An ultrasonic treatment apparatus controller 200 in the ultrasonic treatment apparatus according to the present embodiment has the configuration described with reference to FIG. 16 similarly to the ultrasonic treatment apparatus controller 200 according to the third embodiment, and controls and drives the ultrasonic wave emitter 110 by a method that uses amplitude modulation.

More specifically, for example, a frequency fₘ of modulated waves is stored in an output information storage 215 in association with an object to be irradiated with ultrasonic waves. A frequency f_{c} carrier waves is stored in the output information storage 215 in association with the distance (depth) between an ultrasonic wave emitter 110 and the object to be irradiated with ultrasonic waves. Information regarding the ultrasonic waves emitted by the ultrasonic wave emitter 110, such as the strength of the ultrasonic waves is stored in the output information storage 215. In accordance with an input from an input unit 250, the drive parameter setter 210 reads the information stored in the output information storage 215. On the basis of the read information, the drive parameter setter 210 determines a signal of ultrasonic waves to be emitted from the ultrasonic wave emitter 110, in accordance with the object to be irradiated with ultrasonic waves and the distance to the object. The drive parameter setter 210 outputs, to a drive controller 220, the determined information on the ultrasonic wave signal to be emitted from the ultrasonic wave emitter 110.

On the basis of the signal information input from the drive parameter setter 210, the drive controller 220 outputs an instruction for a carrier wave signal generator 242 to generate a signal, and outputs an instruction for a modulator 244 to modulate the signal. In accordance with the input from the drive controller 220, the carrier wave signal generator 242 generates a carrier wave signal. The carrier wave signal generator 242 outputs the generated signal to the modulator 244. Carrier waves are input to the modulator 244 from the carrier wave signal generator 242. On the basis of the input from the drive controller 220, the modulator 244 generates a modulated wave signal. The modulator 244 modulates, by the generated modulating signal, the carrier waves input from the carrier wave signal generator 242. The modulator 244 outputs a resultant drive signal to the ultrasonic wave emitter 110. In accordance with the drive signal input from the modulator 244, the ultrasonic element 610 that configures the ultrasonic wave emitter 110 generates ultrasonic waves.

In this manner, the ultrasonic wave emitter 110 emits ultrasonic waves in accordance with, for example, the signal x(t) which is output by the modulator 244 and which is represented by, for example, Equation (2). As a result, the ultrasonic waves propagate to a target position on the carrier waves, and the ultrasonic waves having the frequency of the modulated waves are self-demodulated at the target position. That is, the ultrasonic waves having the frequency of the modulated waves are applied to the target position.

According to the ultrasonic treatment apparatus of the present embodiment, ultrasonic waves having a frequency suited for an object can be radiated regardless of the configuration of the ultrasonic wave emitter 110, as in the third embodiment and eighth embodiment. A frequency of the carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

The piezoelectric element 611 of the ultrasonic element 610 has only to vary in thickness from place to place. Therefore, as in the eighth embodiment, the piezoelectric element 611 may be shaped, for example, as shown in FIGS. 31A and 31B. Similar advantages can be obtained in this case as well.

The endoscopic ultrasonic diagnostic treatment apparatus 400 described in the second embodiment can be configured by use of the ultrasonic treatment apparatus according to the ninth embodiment. The endoscopic ultrasonic diagnostic treatment apparatus 400 configured by use of the ultrasonic treatment apparatus according to the ninth embodiment can provide operation and advantages similar to those in the second embodiment.

### Tenth Embodiment

A tenth embodiment of the present invention is described with reference to the drawings. In an ultrasonic treatment apparatus according to the present embodiment, a piezoelectric element that varies in thickness from place to place is used as an ultrasonic wave emitter 110, similarly to the ultrasonic wave emitter 110 according to the eighth embodiment. An ultrasonic treatment apparatus controller 200 in the ultrasonic treatment apparatus according to the present embodiment has the configuration described with reference to FIG. 18 similarly to the ultrasonic treatment apparatus controller 200 according to the fourth embodiment, and controls and drives the ultrasonic wave emitter 110 by a method that uses a parametric effect attributed to a difference tone.

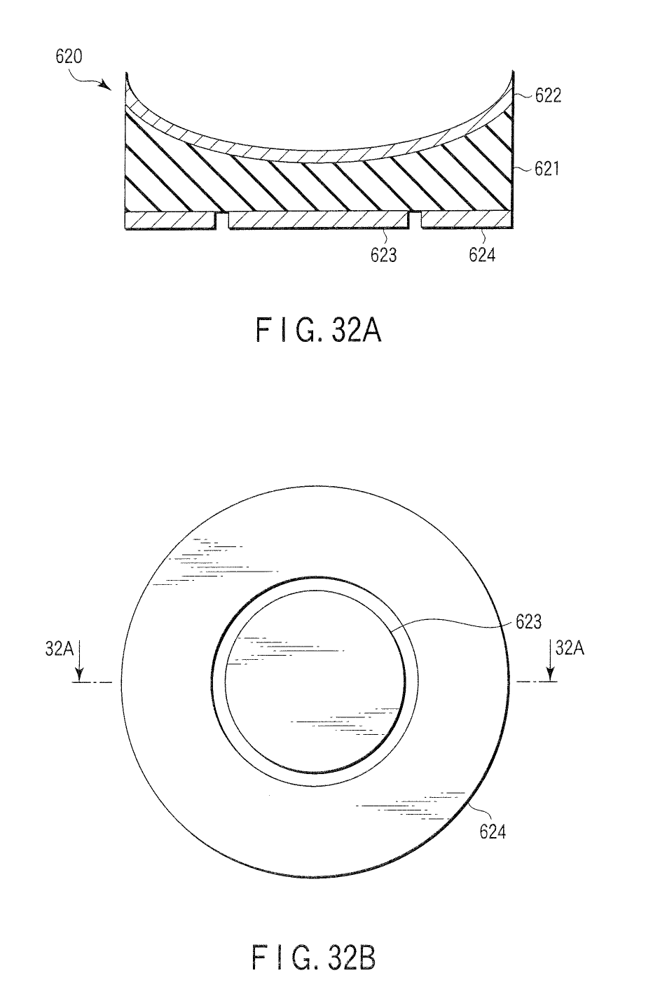

The ultrasonic wave emitter 110 according to the present embodiment includes a plano-concave piezoelectric element 621 having a concave upper surface and a planar lower surface, as shown in a sectional view in FIG. 32A and a plane view of the lower surface in FIG. 32B. The piezoelectric element 621 is different from the ultrasonic element 610 according to the eighth embodiment in the shape of the lower electrode. In an ultrasonic element 620 according to the present embodiment, a ground electrode 622, for example, is formed on the upper surface of the plano-concave piezoelectric element 621. For example, a first electrode 623 and a second electrode 624 are concentrically formed on the lower surface of the piezoelectric element 621. A first signal generator 232 of the ultrasonic treatment apparatus controller 200 is connected to the electrode 623 of the ultrasonic element 620 having the configuration described above. A second signal generator 234 is connected to the second electrode 624. For example, the first electrode 623 functions as at least one drive electrode to which a first drive signal is input. For example, the second electrode 624 functions as at least another drive electrode to which a second drive signal is input.

For example, when the ultrasonic element 620 is formed as shown in FIGS. 32A and 32B, a high-frequency signal is input to the first electrode 623, and a low-frequency signal is input to the second electrode 624. As a result, the central portion of the ultrasonic element 620 vibrates at a frequency input to the first electrode 623, and the peripheral portion vibrates at a frequency input to the second electrode 624.

The ultrasonic treatment apparatus controller 200 operates as in the fourth embodiment. That is, in response to an operator instruction input from an input unit 250, a drive parameter setter 210 of the ultrasonic treatment apparatus controller 200 determines frequencies of carrier waves and modulated waves in accordance with a target position and an object to be irradiated with ultrasonic waves. For the determined frequencies of the carrier waves and the modulated waves, the drive parameter setter 210 determines the frequency f₁, amplitude A₁ and initial phase θ₁ of a signal generated by the first signal generator 232 and the frequency f₂, amplitude A₂ and initial phase θ₂ of a signal generated by the second signal generator 234. Here, the drive parameter setter 210 uses frequency information stored in an output information storage 215. The drive parameter setter 210 outputs the determined f₁, f₂, A₁, A₂, θ₁ and θ₂ to the drive controller 220.

On the basis of the value input from the drive parameter setter 210, the drive controller 220 outputs an instruction for the first signal generator 232 to generate a signal having the frequency f₁, amplitude A₁ and initial phase θ₁. The drive controller 220 also outputs an instruction for the second signal generator 234 to generate a signal having the frequency f₂, amplitude A₂ and initial phase θ₂. The first signal generator 232 outputs the generated signal to the first electrode 623. The second signal generator 234 also outputs the generated signal to the second electrode 624.

As a result, ultrasonic waves are generated from the ultrasonic element 620 by the vibration of the piezoelectric element 621. The generated ultrasonic waves are radiated into a subject. In the subject irradiated with the ultrasonic waves, ultrasonic waves having a frequency Δf=|f₁−f₂| which is a difference tone are generated by the parametric effect as in the fourth embodiment.

According to the ultrasonic treatment apparatus of the present embodiment, ultrasonic waves having a frequency suited for an object can be radiated regardless of the configuration of the ultrasonic wave emitter 110, as in the fourth embodiment and eighth embodiment. A frequency of the carrier waves is properly selected in such a manner as to take the attenuation of ultrasonic waves into account in accordance with the depth of the target position to be irradiated with ultrasonic waves. This ensures that the ultrasonic treatment apparatus can apply ultrasonic waves having the frequency of the set modulated waves to various target positions. Moreover, the use and propagation of the high-frequency ultrasonic waves advantageously allow for a sharper beam pattern.

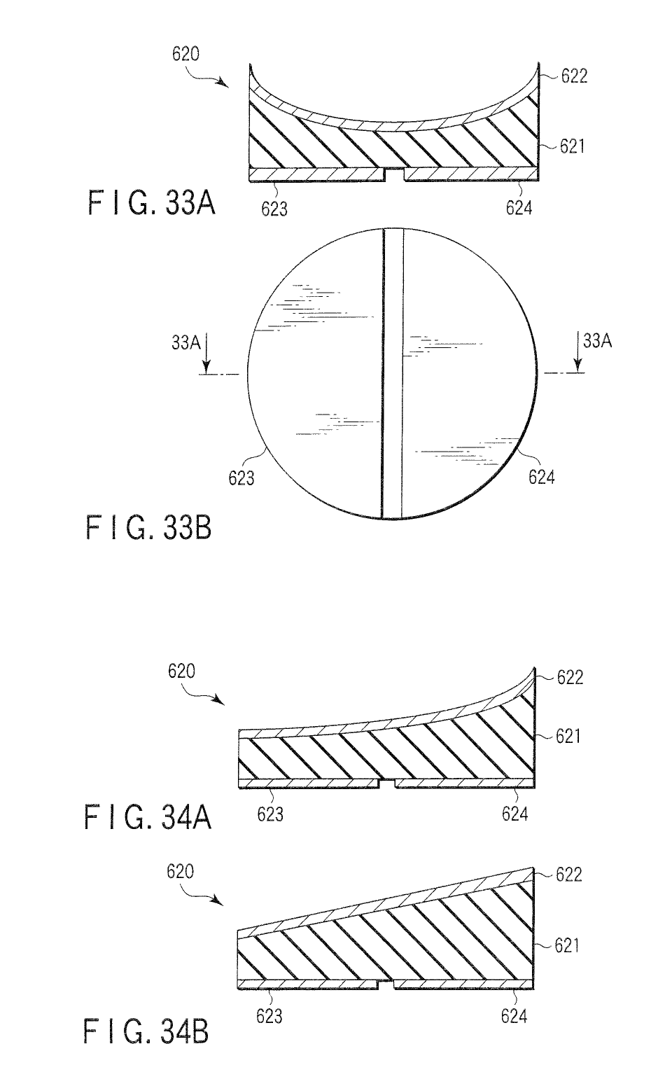

The piezoelectric element 611 of the ultrasonic element 610 has only to vary in thickness from place to place. The first electrode 623 and the second electrode 624 have only to be formed separately. Thus, for example, as shown in FIGS. 33A and 33B, the first electrode 623 and the second electrode 624 may be formed in a halved state. Similar advantages can be obtained in this case as well. Moreover, as in the eighth embodiment, the ultrasonic element 620 may be shaped as shown in FIGS. 34A and 34B. In this case as well, similar advantages can be obtained.

The endoscopic ultrasonic diagnostic treatment apparatus 400 described in the second embodiment can be configured by use of the ultrasonic treatment apparatus according to the tenth embodiment. The endoscopic ultrasonic diagnostic treatment apparatus 400 configured by use of the ultrasonic treatment apparatus according to the tenth embodiment can provide operation and advantages similar to those in the second embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment apparatus comprising:
a sound source which emits ultrasonic waves, the ultrasonic waves being finite amplitude acoustic waves including a first signal frequency and a second signal frequency;
a frequency setter which sets the first signal frequency and the second signal frequency in accordance with a target position of a treatment object with respect to the sound source; and
a drive signal generator configured to generate a drive signal and to drive the sound source, the drive signal being a signal which causes the sound source to emit the ultrasonic waves.

2. The ultrasonic treatment apparatus according to claim 1, further comprising:
a frequency information storage to store information that associates the target position with the first signal frequency and the second signal frequency,
wherein the frequency setter sets the first signal frequency and the second signal frequency based on the information stored in the frequency information storage.

3. The ultrasonic treatment apparatus according to claim 1, further comprising a position information input unit to input the target position.

4. The ultrasonic treatment apparatus according to claim 1, wherein the drive signal generator comprises
a signal generator which generates a first drive signal having the first signal frequency and a second drive signal having the second signal frequency, and
an adder which adds the first drive signal and the second drive signal together to generate an additional drive signal; and
the sound source is driven by the additional drive signal.

5. The ultrasonic treatment apparatus according to claim 1, wherein the drive signal generator generates the drive signal by amplitude modulation.

6. The ultrasonic treatment apparatus according to claim 1, wherein the sound source comprises ultrasonic wave emitters,
the drive signal generator generates a first drive signal having the first signal frequency and a second drive signal having the second signal frequency, and
at least one of the ultrasonic wave emitters is driven by the first drive signal, and at least one of the ultrasonic wave emitters is driven by the second drive signal.

7. The ultrasonic treatment apparatus according to claim 1, wherein the sound source comprises a capacitive transducer.

8. The ultrasonic treatment apparatus according to claim 7, wherein the capacitive transducer includes
a vibrating film, and
a head which is formed on the vibrating film and which brings vibrating directions into one direction.

9. The ultrasonic treatment apparatus according to claim 7, wherein the drive signal generator comprises
a signal generator which generates a first drive signal having the first signal frequency and a second drive signal having the second signal frequency, and
an adder which adds the first drive signal and the second drive signal together to generate an additional drive signal; and
the sound source is driven by the additional drive signal.

10. The ultrasonic treatment apparatus according to claim 7, wherein the drive signal generator generates the drive signal by amplitude modulation.

11. The ultrasonic treatment apparatus according to claim 1, wherein the sound source comprises capacitive transducers, and
each of the capacitive transducers includes
a vibrating film, and
a head which is formed on the vibrating film and which brings vibrating directions into one direction, the head being sloped.

12. The ultrasonic treatment apparatus according to claim 11, wherein the drive signal generator selects the capacitive transducer to be driven by the drive signal among the capacitive transducers based on a slope of the head in accordance with the target position.

13. The ultrasonic treatment apparatus according to claim 1, wherein the sound source comprises capacitive transducers,
the drive signal generator generates a first drive signal having the first signal frequency and a second drive signal having the second signal frequency, and
at least one of the capacitive transducers is driven by the first drive signal, and at least one of the capacitive transducers is driven by the second drive signal.

14. The ultrasonic treatment apparatus according to claim 1, wherein the sound source comprises an interdigital electrode transducer including a piezoelectric substrate and an interdigital electrode formed on the piezoelectric substrate.

15. The ultrasonic treatment apparatus according to claim 14, wherein the interdigital electrode is arc-shaped, and
the ultrasonic waves generated by the interdigital electrode transducer propagates in a direction of a center of the arc.

16. The ultrasonic treatment apparatus according to claim 14, wherein the interdigital electrode is shaped to continuously vary in width in a longitudinal direction of the interdigital electrode.

17. The ultrasonic treatment apparatus according to claim 14, wherein the piezoelectric substrate comprises Y-cut X-propagation lithium niobate.

18. The ultrasonic treatment apparatus according to claim 14, wherein the finite amplitude acoustic waves have a frequency included in a frequency band of surface acoustic waves or bulk waves generated by the interdigital electrode transducer.

19. The ultrasonic treatment apparatus according to claim 14, wherein the interdigital electrode transducer comprises interdigital electrodes, the interdigital electrodes being arranged so that traveling directions of the ultrasonic waves generated from the respective interdigital electrodes intersect with one another.

20. The ultrasonic treatment apparatus according to claim 19, wherein the drive signal generator generates a first drive signal having the first signal frequency and a second drive signal having the second signal frequency, and
the first drive signal is input to at least one of the interdigital electrodes, and the second drive signal is input to at least one of the interdigital electrodes.

21. The ultrasonic treatment apparatus according to claim 14, wherein the drive signal generator comprises
a signal generator which generates a first drive signal having the first signal frequency and a second drive signal having the second signal frequency, and
an adder which adds the first drive signal and the second drive signal together to generate an additional drive signal; and
the sound source is driven by the additional drive signal.

22. The ultrasonic treatment apparatus according to claim 14, wherein the drive signal generator generates the drive signal by amplitude modulation.

23. The ultrasonic treatment apparatus according to claim 1, wherein the sound source comprises a piezoelectric element varying in thickness from place to place.

24. The ultrasonic treatment apparatus according to claim 23, wherein the piezoelectric element is a plano-concave element.

25. The ultrasonic treatment apparatus according to claim 23, wherein the piezoelectric element comprises
a common electrode formed on a first main surface, and
drive electrodes formed on a second main surface opposite to the first main surface on which the common electrode is formed, the drive electrodes being supplied with the drive signal.

26. The ultrasonic treatment apparatus according to claim 25, wherein the drive electrodes are concentrically arranged.

27. The ultrasonic treatment apparatus according to claim 25, wherein the piezoelectric element comprises two drive electrodes formed in the second main surface opposite to the first main surface on which the common electrode is formed, the drive electrodes being supplied with the drive signal.

28. The ultrasonic treatment apparatus according to claim 25, wherein the drive signal generator generates a first drive signal having the first signal frequency and a second drive signal having the second signal frequency, and
the first drive signal is input to at least one of the drive electrodes, and the second drive signal is input to at least one of the drive electrodes.

29. The ultrasonic treatment apparatus according to claim 23, wherein the drive signal generator comprises
a signal generator which generates a first drive signal having the first signal frequency and a second drive signal having the second signal frequency, and
an adder which adds the first drive signal and the second drive signal together to generate an additional drive signal; and
the sound source is driven by the additional drive signal.

30. The ultrasonic treatment apparatus according to claim 23, wherein the drive signal generator generates the drive signal by amplitude modulation.

31. The ultrasonic treatment apparatus according to claim 1, further comprising an image acquisition signal setter which sets an image acquisition emission ultrasonic wave signal for ultrasonic image acquisition by emitting imaging ultrasonic waves which are finite amplitude acoustic waves having the frequency of the image acquisition emission ultrasonic wave signal and by receiving reflected waves,
wherein in the ultrasonic image acquisition, the drive signal generator generates a drive signal causing the sound source to emit the imaging ultrasonic waves, instead of the drive signal causing the sound source to emit the ultrasonic waves which are the finite amplitude acoustic waves including the first signal frequency and the second signal frequency.
